(19) United States
(12) Patent Application Publication
(10) Pub. No.: US 2011/0028573 A1
(43) Pub. Date: Feb. 3, 2011

(54) High Shear Production of Value-Added Product From Refinery-Related Gas

(75) Inventors: Abbas HASSAN, Sugar Land, TX (US); Aziz HASSAN, Sugar Land, TX (US); Rayford G. ANTHONY, College Station, TX (US); Gregory G. BORSINGER, Chatham, NJ (US)

Correspondence Address: CONLEY ROSE, P.C.;David A. Rose, P. O. BOX 3267, HOUSTON, TX, 77253-3267, US

(73) Assignee: HRD CORP., Houston, TX (US)

(21) Appl. No.: 12/844,723

(22) Filed: Jul. 27, 2010

Related U.S. Application Data
(60) Provisional application No. 61/229,082, filed on Jul. 28, 2009.

(51) Int. Cl. C07C 27/06; C07C 27/16; B01J 10/00

(52) U.S. Cl. 518/700; 208/3; 422/604

(57) ABSTRACT

A method of producing value-added product from refinery-related gas, the method comprising: providing a refinery-related gas comprising at least one selected from C1-C8 compounds; intimately mixing the refinery-related gas with a liquid carrier in a high shear device to form a dispersion of gas in the liquid carrier, wherein the gas bubbles in the dispersion have a mean diameter of less than or equal to about 5 μm; and extracting value-added product comprising at least one component selected from higher hydrocarbons, olefins and alcohols. A system for producing value-added product from refinery-related gas comprising: at least one high shear device comprising at least one rotor and at least one complementarily-shaped stator; apparatus for the production of a refinery-related gas comprising one or more of C1-C8 compounds; and a pump configured for delivering a liquid stream comprising the liquid carrier to the high shear device.

### CROSS-REFERENCE TO RELATED APPLICATIONS

This application claims the benefit under 35 U.S.C. §119(e) of U.S. Provisional Patent Application No. 61/229,082, filed Jul. 28, 2009, the disclosure of which is hereby incorporated herein by reference.

### STATEMENT REGARDING FEDERALLY SPONSORED RESEARCH OR DEVELOPMENT

Not Applicable.

### BACKGROUND

1. Technical Field

The present invention relates generally to production of value-added products from refinery-related gas. More particularly, the present invention relates to an apparatus and process for producing product comprising oxygenate(s) via shear-promoted reaction of refinery-related gas.

2. Background of the Invention

Oil refineries are utilized for processing crude oil and refining it into more useful petroleum products, such as gasoline, diesel fuel, asphalt base, heating oil, kerosene, and liquefied petroleum gas. Oil refineries are typically large sprawling industrial complexes with extensive piping running throughout, carrying streams of fluids between large chemical processing units.

Many of the processes utilized in oil refineries create large quantities of gas. A substantial quantity of this gas is negative-value gas, i.e. there is financial loss incurred in disposing of the gas. Much of the gas produced in a refinery is sent to a gas plant which serves to create value-added products or otherwise treat the gas before its use as a fuel gas or flaring of the gas to the environment. Flaring may be undesirable due to environmental regulations. Additionally, crude oil is often discovered with associated gas which is generally separated therefrom prior to refining of the crude oil.

Accordingly, there is a need in industry for systems and processes of converting refinery-related gas into value-added products. Desirably, the conversion is such that the conversion of the refinery-related gas to value-added product is economically beneficial. Desirably, the system and process may be incorporated into existing refineries or designed into the building of new refineries. There is also a need for systems and processes for enhancing the API of crude oil and/or increasing the stability of crude oil. Elimination of catalyst entirely is also possible in some instances.

### SUMMARY

Herein disclosed is a method of producing value-added product from refinery-related gas, the method comprising: providing a refinery-related gas comprising at least one compound selected from the group consisting of C1-C8 compounds and combinations thereof; (b) intimately mixing the refinery-related gas with a liquid carrier in a high shear device to form a dispersion of gas in the liquid carrier, wherein the gas bubbles in the dispersion have a mean diameter of less than or equal to about 5 micron(s); and (c) extracting value-added product comprising at least one component selected from the group consisting of higher hydrocarbons, olefins, alcohols, aldehydes, and ketones. In some cases, the refinery-related gas is selected from the group consisting of pyrolysis gas, FCC offgas, associated gas, hydrodesulfurization offgas, coker offgas, catalytic cracker offgas, thermal cracker offgas, and combinations thereof. In some cases, the C1-C8 compounds comprise carbon dioxide.

In an embodiment, alcohol as the value-added product is selected from the group consisting of methanol, ethanol, isopropanol, butanol, and propanol. In an embodiment, step (b) further comprises contacting the refinery-related gas and the carrier with a catalyst. In some cases, the catalyst comprises at least one component selected from the group consisting of phosphoric acid, sulfonic acid, sulfuric acid, zeolites, solid acid catalysts, and liquid acid catalysts. In some embodiments, the carrier is a catalyst. In some embodiments, the carrier comprises sulfuric acid. In some embodiments, the carrier comprises water. In an embodiment, step (c) comprises separating a light gas from the carrier and the value-added product. In another embodiment, the method further comprises contacting the carrier and the refinery-related gas with a catalyst selected from the group consisting of hydrogenation catalysts, hydroxylation catalysts, partial oxidation catalysts, hydrodesulfurization catalysts, hydrodenitrogenation catalysts, hydrofinishing catalysts, reforming catalysts, hydration catalysts, hydrocracking catalysts, Fischer-Tropsch catalysts, dehydrogenation catalysts, and polymerization catalysts.

In an embodiment, a method of increasing the API gravity of a crude oil is described. The method comprises: introducing a crude oil and a gas selected from the group consisting of oxygenates, associated gas, unassociated gas, light gas separated from the carrier and the value-added product, and combinations thereof into a high shear device comprising at least one rotor and at least one stator; and rotating the rotor to provide a tip speed of at least 22.9 m/s. In some embodiments, the API gravity is increased by a factor of at least 1.5.

Further described in this disclosure is a system for producing value-added product from refinery-related gas, the system comprising: at least one high shear device comprising at least one rotor and at least one complementarily-shaped stator, configured to produce a dispersion comprising bubbles of refinery-related gas in a liquid carrier; apparatus for the production of a refinery-related gas comprising one or more of C1-C8 compounds; and a pump configured for delivering a liquid stream comprising the liquid carrier to the high shear device. In some embodiments, the system further comprises a vessel coupled to the high shear device, the vessel configured for receiving the dispersion from the high shear device.

In an embodiment, the at least one rotor is rotatable at a tip speed of at least 22.9 m/s (4,500 ft/min), wherein the tip speed is defined as πDn, where D is the diameter of the rotor and n is the frequency of revolution. In an embodiment, the at least one rotor is separated from the at least one stator by a shear gap in the range of from in the range of from about 0.02 mm to about 5 mm, wherein the shear gap is the minimum distance between the at least one rotor and the at least one stator. In an embodiment, the at least one rotor is able to provide shear rate of at least 20,000 s⁻¹ during operation, wherein the shear rate is defined as the tip speed divided by the shear gap, and wherein the tip speed is defined as πDn, where D is the diameter of the rotor and n is the frequency of revolution.

In an embodiment, the system comprises more than one high shear device. In an embodiment, the high shear device comprises at least two generators, wherein each generator comprises a rotor and a complementarily-shaped stator. In an embodiment, the apparatus for the production of refinery-related gas comprises a cracker configured for breaking organic molecules into simpler molecules. In an embodiment, the apparatus for the production of refinery-related gas comprises an oil refinery or some components thereof, a fossil fuel burning facility or some components thereof. In an embodiment, the fossil fuel burning facility is a power plant or a power station.

Herein disclosed is a method of producing value-added product from refinery-related gas, the method comprising: (a) providing a refinery-related gas comprising at least one selected from primarily C1-C8 compounds and hydrogen; (b) intimately mixing the refinery-related gas with a liquid carrier in a high shear device to form a dispersion of gas in the liquid carrier, wherein the gas bubbles in the dispersion have a mean diameter of less than or equal to about 5 micron(s); and (c) extracting value-added product comprising at least one component selected from higher hydrocarbons, olefins and alcohols. In embodiments, the gas bubbles have an average diameter of no more than about 5, 4, 3, 2, 1, 0.5, 0.4, 0.3, 0.2, or 0.1 μm. In embodiments, the gas bubbles have an average diameter of no more than about 100 nm. The refinery-related gas can be selected from pyrolysis gas, FCC offgas, associated gas, hydrodesulfurization offgas, coker offgas, catalytic cracker offgas, thermal cracker offgas, or other hydrocarbon processing or combustion sources and combinations thereof. In embodiments, the high shear device comprises at least one rotor and at least one stator and (b) comprises subjecting the gas-liquid stream to high shear mixing at a tip speed of at least about 23 msec, wherein the tip speed is defined as πDn, where D is the diameter of the at least one rotor and n is the frequency of revolution. In embodiments, the high shear device comprises at least one rotor and at least one stator, and (b) comprises providing a shear rate of at least 20,000 s⁻¹, wherein the shear rate is defined as the tip speed divided by the shear gap, and wherein the tip speed is defined as πDn, where D is the diameter of the at least one rotor and n is the frequency of revolution. Providing a shear rate of at least 20,000 s⁻¹ may produce a local pressure of at least about 1034.2 MPa (150,000 psi) at a tip of the at least one rotor. Providing a shear rate of at least 20,000 s⁻¹ may comprise rotating the at least one rotor at a tip speed of at least 22.9 m/s (4,500 ft/min), wherein the tip speed is defined as πDn, where D is the diameter of the rotor and n is the frequency of revolution. Forming the dispersion can comprise an energy expenditure of at least about 1000 W/m³, 5000 W/m³, 7500 W/m³, 1 kW/m³, 500 kW/m³, 1000 kW/m³, 5000 kW/m³, 7500 kW/m³, or greater.

In embodiments, (b) further comprises contacting the refinery-related gas and the carrier with a catalyst. The catalyst may be selected from solid acid catalysts and liquid catalysts. The catalyst may be selected from phosphoric acid, sulfonic acid, sulfuric acid, zeolites, hydrosilane and combinations thereof. Catalyst may also contain a noble metal such as nickel, ruthenium, rhodium, or platinum as an active component. Biocatalysts may also be utilized. In embodiments, the carrier is a catalyst. In embodiments, the carrier comprises sulfuric acid. In embodiments, the alcohol(s) produced comprises at least one selected from methanol, ethanol, isopropanol, butanol, and propanol.

In embodiments, (c) comprises separating a light gas from the carrier and the value-added product. The method may further comprise subjecting the light gas to high shear. Subjecting the light gas to high shear may comprise introducing the light gas into a high shear device comprising at least one rotor and at least one stator in the presence of a Fischer-Tropsch catalyst, whereby Fischer-Tropsch hydrocarbons are produced. Subjecting the light gas to high shear may comprise providing a shear rate of at least 20,000 s⁻¹, wherein the shear rate is defined as the tip speed divided by the shear gap, and wherein the tip speed is defined as πDn, where D is the diameter of the at least one rotor and n is the frequency of revolution. Subjecting the light gas to high shear may comprise introducing the light gas and crude oil into a high shear device comprising at least one rotor and at least one stator, and subjecting the contents of the high shear device to a shear rate of at least 20,000 s⁻¹. In various embodiments, high shear is applied to the light gas together with a liquid or slurry.

The method may further comprise contacting the carrier (a liquid or slurry) and the refinery-related gas to a catalyst selected from the group consisting of hydrogenation catalysts, hydroxylation catalysts, partial oxidation catalysts, hydrodesulfurization catalysts, hydrodenitrogenation catalysts, hydrofinishing catalysts, reforming catalysts, hydration catalysts, hydrocracking catalysts, Fischer-Tropsch catalysts, dehydrogenation catalysts, and polymerization catalysts.

Also disclosed is a method of increasing the API gravity of a crude oil, the method comprising: introducing the crude oil and a gas selected from oxygenates, associated gas, unassociated gas, light gas from the above-disclosed method of producing value-added product from refinery-related gas, or a combination thereof into a high shear device comprising at least one rotor and at least one stator; and rotating the rotor to provide a tip speed of at least 22.9 m/s. In embodiments, the API gravity is increased by a factor of at least 1.5. In embodiments, the API gravity is increased by a factor of at least 2.

Also disclosed is a system for producing value-added product from refinery-related gas, the system comprising: at least one high shear device comprising at least one rotor and at least one complementarily-shaped stator, configured to produce a dispersion comprising bubbles of refinery-related gas in a liquid carrier; apparatus for the production of a refinery-related gas comprising one or more of C1-C8 compounds and hydrogen; and a pump configured for delivering a liquid stream comprising the liquid carrier to the high shear device. The system may further comprise a vessel coupled to the high shear device, the vessel configured for receiving the dispersion from the high shear device. In embodiments, the at least one rotor is rotatable at a tip speed of at least 22.9 m/s (4,500 ft/min), wherein the tip speed is defined as πDn, where D is the diameter of the rotor and n is the frequency of revolution. In embodiments, the high shear device is configured for operating at a tip speed of at least 40 msec. In embodiments, the at least one rotor is separated from the at least one stator by a shear gap in the range of from about 0.02 mm to about 5 mm, wherein the shear gap is the minimum distance between the at least one rotor and the at least one stator. In embodiments, the shear rate provided by rotation of the at least one rotor during operation is at least 20,000 s⁻¹, wherein the shear rate is defined as the tip speed divided by the shear gap, and wherein the tip speed is defined as πDn, where D is the diameter of the rotor and n is the frequency of revolution. In embodiments, the high shear device comprises two or more rotors and two or more stators. In embodiments, the at least one high shear device is configured for producing a dispersion of bubbles of refinery-related gas in the liquid phase, wherein the dispersion has a mean bubble diameter of less than about 5, 4, 3, 2, 1, 0.5, 0.4, 0.3, 0.2, or 0.1 μm. The system can comprise more than one high shear device. In embodiments, the high shear device comprises at least two generators, wherein each generator comprises a rotor and a complementarily-shaped stator. The shear rate provided by one generator may be greater than the shear rate provided by another generator.

In embodiments, the apparatus for the production of refinery-related gas comprises a cracker configured for breaking organic molecules into simpler molecules. The cracker may comprise a fluid catalytic cracker. The cracker may comprise a thermal cracker. The thermal cracker may comprise a coker. In embodiments, the apparatus for the production of refinery-related gas comprises a steam cracker. In embodiments, the apparatus for the production of refinery-related gas comprises an oil refinery or some components thereof.

Also disclosed is a system for producing value-added product from FCC offgas, the system comprising: at least one high shear device comprising at least one rotor and at least one complementarily-shaped stator, wherein the at least one rotor is rotatable at a tip speed of at least 22.9 m/s (4,500 ft/min), wherein the tip speed is defined as πDn, where D is the diameter of the rotor and n is the frequency of revolution; fluid catalytic cracking apparatus configured for the catalytic cracking of a FCC feedstock and operable to produce a FCC offgas, the at least one high shear device in fluid communication with a line configured for carrying at least a portion of the FCC offgas to the at least one high shear device; and a pump configured for delivering a liquid stream comprising liquid carrier to the high shear device. In embodiments, the FCC feedstock comprises AGO, VGO, light vacuum distillate, heavy vacuum distillate, or a combination thereof. The system may further comprise a fluid catalytic cracking vapor recovery unit configured for separating at least one component from the FCC offgas. In embodiments, the high shear device is operable to produce a dispersion of the FCC offgas in the liquid carrier, wherein the bubbles of FCC offgas in the dispersion have an average bubble diameter of less than 5 microns. In embodiments, the bubbles of FCC offgas in the dispersion have a bubble diameter of less than 5, 4, 3, 2, 1, 0.5, 0.4, 0.3, 0.2, or 0.1 μm. In embodiments, the at least one rotor is rotatable at a tip speed of at least 40 m/s.

Also disclosed is a system for producing value-added product from coker offgas, the system comprising: at least one high shear device comprising at least one rotor and at least one complementarily-shaped stator, wherein the at least one rotor is rotatable at a tip speed of at least 22.9 m/s (4,500 ft/min), wherein the tip speed is defined as πDn, where D is the diameter of the rotor and n is the frequency of revolution; a coker configured for thermal cracking of a coker feedstock and operable to produce a coker offgas, the at least one high shear device in fluid communication with a line configured for carrying at least a portion of the coker offgas to the at least one high shear device; and a pump configured for delivering a liquid stream comprising liquid carrier to the high shear device. In embodiments, the coker is a delayed coker. In embodiments, the coker feedstock comprises residual. In embodiments, the high shear device is operable to produce a dispersion of the coker offgas in the liquid carrier, wherein the bubbles of coker offgas in the dispersion have an average bubble diameter of less than 5, 4, 3, 2, 1, 0.5, 0.4, 0.3, 0.2, or 0.1 μm. In embodiments, the bubbles of coker offgas in the dispersion have a bubble diameter of less than 1 micron. In embodiments, the at least one rotor is rotatable at a tip speed of at least 40 m/s.

Also disclosed is a system for producing value-added product from pyrolysis, the system comprising: at least one high shear device comprising at least one rotor and at least one complementarily-shaped stator, wherein the at least one rotor is rotatable at a tip speed of at least 22.9 m/s (4,500 ft/min), wherein the tip speed is defined as πDn, where D is the diameter of the rotor and n is the frequency of revolution; a steam cracker configured for producing pyrolysis gas from a feedstock, the at least one high shear device in fluid communication with a line configured for carrying at least a portion of the pyrolysis gas to the at least one high shear device; and a pump configured for delivering a liquid stream comprising liquid carrier to the high shear device. In embodiments, the system further comprises a separator upstream of the at least one high shear device for separating at least one component from the pyrolysis gas. In embodiments, the steam cracker feedstock comprises naphtha. In embodiments, the high shear device is operable to produce a dispersion of the pyrolysis gas in the liquid carrier, wherein the bubbles of pyrolysis gas in the dispersion have an average bubble diameter of less than 5, 4, 3, 2, 1, 0.5, 0.4, 0.3, 0.2, or 0.1 μm. In embodiments, the bubbles of pyrolysis gas in the dispersion have a bubble diameter of less than 1 micron. In embodiments, the at least one rotor is rotatable at a tip speed of at least 40 m/s.

Certain embodiments of the above-described methods or systems potentially provide overall cost reduction by providing for reduced catalyst usage, permitting increased fluid throughput, permitting operation at lower temperature and/or pressure, and/or reducing capital and/or operating costs. These and other embodiments and potential advantages will be apparent in the following detailed description and drawings.

### BRIEF DESCRIPTION OF THE DRAWINGS

For a more detailed description of the preferred embodiment of the present invention, reference will now be made to the accompanying drawings, wherein:

FIG. 1 is a schematic of a high shear system according to an embodiment of the present disclosure comprising external high shear dispersing.

FIG. 2 is a longitudinal cross-section view of a high shear mixing device suitable for use in embodiments of the system of FIG. 1.

FIG. 3 is a schematic of a suitable Refinery-Related Gas (RRG) production apparatus 15A according to an embodiment of this disclosure.

FIG. 4 is a schematic of a suitable RRG production apparatus 15B according to an embodiment of this disclosure.

FIG. 5 is a schematic of a suitable RRG production apparatus 15C according to an embodiment of this disclosure.

FIG. 6 is a schematic of a suitable RRG production apparatus 15D according to an embodiment of this disclosure.

FIG. 7 is a schematic of a suitable RRG production apparatus 15E according to an embodiment of this disclosure.

FIG. 8 is a diagram of a method of producing value product from RRG 250 according to an embodiment of this disclosure.

FIG. 9 is a box diagram of a method of providing RRG 300A according to an embodiment of this disclosure.

FIG. 10 is a schematic of a method of providing cracker feedstock 301A according to an embodiment of this disclosure.

FIG. 11 is a box diagram of a method of providing RRG 300B according to an embodiment of this disclosure.

FIG. 12 is a box diagram of a method of providing RRG 300C according to an embodiment of this disclosure.

FIG. 13 is box diagram of a method of providing RRG 300D according to an embodiment of this disclosure.

FIG. 14 is a box diagram of a method 600A of adjusting the stability and or the API gravity of crude oil according to an embodiment of this disclosure.

### NOTATION AND NOMENCLATURE

As used herein, the term ‘dispersion’ refers to a liquefied mixture that contains at least two distinguishable substances (or ‘phases’) that will not readily mix and dissolve together. As used herein, a ‘dispersion’ comprises a ‘continuous’ phase (or ‘matrix’), which holds therein discontinuous droplets, bubbles, and/or particles of the other phase or substance. The term dispersion may thus refer to foams comprising gas bubbles suspended in a liquid continuous phase, emulsions in which droplets of a first liquid are dispersed throughout a continuous phase comprising a second liquid with which the first liquid is immiscible, and continuous liquid phases throughout which solid particles are distributed. As used herein, the term “dispersion” encompasses continuous liquid phases throughout which gas bubbles are distributed, continuous liquid phases throughout which solid particles (e.g., solid catalyst) are distributed, continuous phases of a first liquid throughout which droplets of a second liquid that is substantially insoluble in the continuous phase are distributed, and liquid phases throughout which any one or a combination of solid particles, immiscible liquid droplets, and gas bubbles are distributed. Hence, a dispersion can exist as a homogeneous mixture in some cases (e.g., liquid/liquid phase), or as a heterogeneous mixture (e.g., gas/liquid, solid/liquid, or gas/solid/liquid), depending on the nature of the materials selected for combination.

The use of the term ‘refinery-related gas,’ or the acronym ‘RRG,’ is intended to refer to any suitable gas obtained in an oil refinery or obtained from extraction of crude oil or and/or associated gas from the earth. Generally, the RRG comprises at least one of C1 to C8 compounds and may contain hydrogen. In embodiments, the RRG comprises at least one of C1 to C4 compounds and may contain hydrogen. For example, the RRG can comprise one or more chosen from methane, ethane, propane, butane, ethylene, propylene, butylene, carbon dioxide, carbon monoxide, and hydrogen.

The term ‘gas oil’ refers to middle-distillate petroleum fraction with a boiling range of about 350° F. to 750° F., and may include diesel fuel, kerosene, heating oil, and light fuel oil.

The term ‘gasoline’ refers to a blend of naphthas and other refinery products with sufficiently high octane and other desirable characteristics to be suitable for use as fuel in internal combustion engines.

Use of the phrase, ‘all or a portion of’ is used herein to mean ‘all or a percentage of the whole’ or ‘all or some components of.’

### DETAILED DESCRIPTION

Overview. A system and process for producing value-added products from refinery-related gas (hereinafter RRG) comprises an external high shear mechanical device to provide rapid contact and mixing of reactants in a controlled environment in the reactor/mixer device. A reactor assembly that comprises an external high shear device (HSD) or mixer as described herein may decrease mass transfer limitations and thereby allow the reaction, which may be catalytic, to more closely approach kinetic and/or thermodynamic limitations. Enhanced mixing may also homogenize the temperature within the reaction zone(s). Enhancing contact via the use of high shear may permit increased throughput and/or the use of a decreased amount of catalyst relative to conventional processes. The use of a HSD may also provide for eliminating the use of catalyst entirely in some instances.

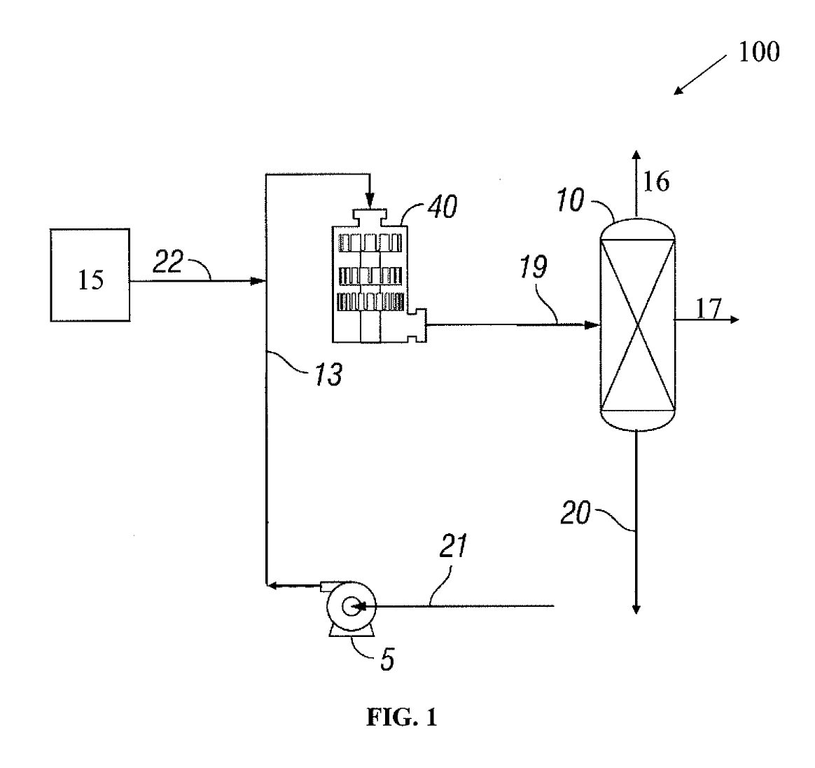

High Shear System for Producing Value-Added Products from Refinery-Related Gas. A high shear system 100 for producing value-added products from refinery-related gas will be described with reference to FIG. 1, which is a process flow diagram of an embodiment of a high shear system 100. The basic components of a representative system include external high shear device (HSD) 40 and pump 5. Each of these components is further described in more detail below. Line 21 is connected to pump 5 for introducing reactants into pump 5. Line 13 connects pump 5 to HSD 40, and line 19 carries product dispersion out of HSD 40. Additional components or process steps can be incorporated between flow line 19 and HSD 40, or ahead of pump 5 or HSD 40, if desired, as will become apparent upon reading the description of the high shear process hereinbelow. For example, line 20 can be connected to line 21 or line 13 from flow line 19 or reactor 10, such that fluid in flow line 19 or from vessel 10 may be recycled to HSD 40. Product may be removed from system 100 via flow line 19. Flow line 19 is any line into which product dispersion (comprising at least liquids and gases) and any unreacted reactants from HSD 40 flow.

System 100 may further comprise a vessel 10 and apparatus for production of RRG 15, as described further hereinbelow. Line 22 is configured to introduce dispersible gas (i.e., RRG) into HSD 40. Line 22 may introduce dispersible gas into HSD directly or may introduce RRG into line 13. In embodiments, line 22 is connected with RRG production apparatus 15. Alternatively, dispersible gas inlet line 22 is connected to an RRG gas storage unit.

High Shear Device. External high shear device (HSD) 40, also sometimes referred to as a high shear mixer, is configured for receiving an inlet stream, via line 13, comprising reactants. Alternatively, HSD 40 may be configured for receiving the reactants via separate inlet lines. Although only one HSD is shown in FIG. 1, it should be understood that some embodiments of the system can comprise two or more HSDs arranged either in series or parallel flow.

HSD 40 is a mechanical device that utilizes one or more generator comprising a rotor/stator combination, each of which has a gap between the stator and rotor. The gap between the rotor and the stator in each generator set may be fixed or may be adjustable. HSD 40 is configured in such a way that it is capable of effectively contacting the reactants with the catalyst therein at rotational velocity. The HSD comprises an enclosure or housing so that the pressure and temperature of the fluid therein may be controlled.

High shear mixing devices are generally divided into three general classes, based upon their ability to mix fluids. Mixing is the process of reducing the size of particles or inhomogeneous species within the fluid. One metric for the degree or thoroughness of mixing is the energy density per unit volume that the mixing device generates to disrupt the fluid particles. The classes are distinguished based on delivered energy densities. Three classes of industrial mixers having sufficient energy density to consistently produce mixtures or emulsions with particle sizes in the range of submicron to 50 microns include homogenization valve systems, colloid mills and high speed mixers. In the first class of high energy devices, referred to as homogenization valve systems, fluid to be processed is pumped under very high pressure through a narrow-gap valve into a lower pressure environment. The pressure gradients across the valve and the resulting turbulence and cavitation act to break-up any particles in the fluid. These valve systems are most commonly used in milk homogenization and can yield average particle sizes in the submicron to about 1 micron range.

At the opposite end of the energy density spectrum is the third class of devices referred to as low energy devices. These systems usually have paddles or fluid rotors that turn at high speed in a reservoir of fluid to be processed, which in many of the more common applications is a food product. These low energy systems are customarily used when average particle sizes of greater than 20 microns are acceptable in the processed fluid.

Between the low energy devices and homogenization valve systems, in terms of the mixing energy density delivered to the fluid, are colloid mills and other high speed rotor-stator devices, which are classified as intermediate energy devices. A typical colloid mill configuration includes a conical or disk rotor that is separated from a complementary, liquid-cooled stator by a closely-controlled rotor-stator gap, which is commonly between 0.025 mm to 10 mm (0.001-0.40 inch). Rotors are usually driven by an electric motor through a direct drive or belt mechanism. As the rotor rotates at high rates, it pumps fluid between the outer surface of the rotor and the inner surface of the stator, and shear forces generated in the gap process the fluid. Many colloid mills with proper adjustment achieve average particle sizes of 0.1 to 25 microns in the processed fluid. These capabilities render colloid mills appropriate for a variety of applications including colloid and oil/water-based emulsion processing such as that required for cosmetics, mayonnaise, or silicone/silver amalgam formation, to roofing-tar mixing.

The HSD comprises at least one revolving element that creates the mechanical force applied to the reactants therein. The HSD comprises at least one stator and at least one rotor separated by a clearance. For example, the rotors can be conical or disk shaped and can be separated from a complementarily-shaped stator. In embodiments, both the rotor and stator comprise a plurality of circumferentially-spaced rings having complementarily-shaped tips. A ring may comprise a solitary surface or tip encircling the rotor or the stator. In embodiments, both the rotor and stator comprise more than 2 circumferentially-spaced rings, more than 3 rings, or more than 4 rings. For example, in embodiments, each of three generators comprises a rotor and stator each having 3 complementary rings, whereby the material processed passes through 9 shear gaps or stages upon traversing HSD 40. Alternatively, each of three generators may comprise four rings, whereby the processed material passes through 12 shear gaps or stages upon passing through HSD 40. In some embodiments, the stator(s) are adjustable to obtain the desired shear gap between the rotor and the stator of each generator (rotor/stator set). Each generator may be driven by any suitable drive system configured for providing the desired rotation.

In some embodiments, HSD 40 comprises a single stage dispersing chamber (i.e., a single rotor/stator combination; a single high shear generator). In some embodiments, HSD 40 is a multiple stage inline disperser and comprises a plurality of generators. In certain embodiments, HSD 40 comprises at least two generators. In other embodiments, HSD 40 comprises at least 3 generators. In some embodiments, HSD 40 is a multistage mixer whereby the shear rate (which varies proportionately with tip speed and inversely with rotor/stator gap width) varies with longitudinal position along the flow pathway, as further described hereinbelow.

According to this disclosure, at least one surface within HSD 40 may be made of, impregnated with, or coated with a catalyst suitable for catalyzing a desired reaction, as described in U.S. patent application Ser. No. 12/476,415, which is hereby incorporated herein by reference for all purposes not contrary to this disclosure. For example, in embodiments, all or a portion of at least one rotor, at least one stator, or at least one rotor/stator set (i.e., at least one generator) is made of, coated with, or impregnated with a suitable catalyst. In some applications, it may be desirable to utilize two or more different catalysts. In such instances, a generator may comprise a rotor made of, impregnated with, or coated with a first catalyst material, and the corresponding stator of the generator may be made of, coated with, or impregnated by a second catalyst material. Alternatively one or more rings of the rotor may be made from, coated with, or impregnated with a first catalyst, and one or more rings of the rotor may be made from, coated with, or impregnated by a second catalyst. Alternatively one or more rings of the stator may be made from, coated with, or impregnated with a first catalyst, and one or more rings of the stator may be made from, coated with, or impregnated by a second catalyst. All or a portion of a contact surface of a stator, rotor, or both can be made from or coated with catalytic material.

A contact surface of HSD 40 can be made from a porous sintered catalyst material, such as platinum. In embodiments, a contact surface is coated with a porous sintered catalytic material. In applications, a contact surface of HSD 40 is coated with or made from a sintered material and subsequently impregnated with a desired catalyst. The sintered material can be a ceramic or can be made from metal powder, such as, for example, stainless steel or pseudoboehmite. The pores of the sintered material may be in the micron or the submicron range. The pore size can be selected such that the desired flow and catalytic effect are obtained. Smaller pore size may permit improved contact between fluid comprising reactants and catalyst. By altering the pore size of the porous material (ceramic or sintered metal), the available surface area of the catalyst can be adjusted to a desired value. The sintered material may comprise, for example, from about 70% by volume to about 99% by volume of the sintered material or from about 80% by volume to about 90% by volume of the sintered material, with the balance of the volume occupied by the pores.

In embodiments, the rings defined by the tips of the rotor/stator contain no openings (i.e. teeth or grooves) such that substantially all of the reactants are forced through the pores of the sintered material, rather than being able to bypass the catalyst by passing through any openings or grooves which are generally present in conventional dispersers. In this manner, for example, a reactant will be forced through the sintered material, thus forcing contact with the catalyst.

In embodiments, the sintered material of which the contact surface is made comprises stainless steel or bronze. The sintered material (sintered metal or ceramic) may be passivated. A catalyst may then be applied thereto. The catalyst may be applied by any means known in the art. The contact surface may then be calcined to yield the metal oxide (e.g. stainless steel). The first metal oxide (e.g., the stainless steel oxide) may be coated with a second metal and calcined again. For example, stainless steel oxide may be coated with aluminum and calcined to produce aluminum oxide. Subsequent treatment may provide another material. For example, the aluminum oxide may be coated with silicon and calcined to provide silica. Several calcining/coating steps may be utilized to provide the desired contact surface and catalyst(s). In this manner, the sintered material which either makes up the contact surface or coats the contact surface may be impregnated with a variety of catalysts. Another coating technique, for example, is metal vapor deposition or chemical vapor deposition, such as typically used for coating silicon wafers with metal.

In embodiments, a sintered metal contact surface (e.g., of the rotor or the stator) is treated with a material. For example, tetra ethyl ortho silicate (TEOS). Following vacuum evaporation, TEOS may remain in surface pores. Calcination may be used to convert the TEOS to silica. This impregnation may be repeated for all desired metal catalysts. Upon formation, coating, or impregnation, the catalyst(s) may be activated according to manufacturer's protocol. For example, catalysts may be activated by contacting with an activation gas, such as hydrogen. The base material may be silicon or aluminum which, upon calcination, is converted to alumina or silica respectively. Suitable catalysts, including without limitation, rhenium, palladium, rhodium, etc. can subsequently be impregnated into the pores.

In some embodiments, the minimum clearance (shear gap width) between the stator and the rotor is in the range of from about 0.025 mm (0.001 inch) to about 3 mm (0.125 inch). In some embodiments, the minimum clearance (shear gap width) between the stator and the rotor is in the range of from about 1 μm (0.00004 inch) to about 3 mm (0.012 inch). In some embodiments, the minimum clearance (shear gap width) between the stator and the rotor is less than about 10 μm (0.0004 inch), less than about 50 μm (0.002 inch), less than about 100 μm (0.004 inch), less than about 200 λm (0.008 inch), less than about 400 μm (0.016 inch). In certain embodiments, the minimum clearance (shear gap width) between the stator and rotor is about 1.5 mm (0.06 inch). In certain embodiments, the minimum clearance (shear gap width) between the stator and rotor is about 0.2 mm (0.008 inch). In certain configurations, the minimum clearance (shear gap) between the rotor and stator is at least 1.7 mm (0.07 inch). The shear rate produced by the HSD may vary with longitudinal position along the flow pathway. In some embodiments, the rotor is set to rotate at a speed commensurate with the diameter of the rotor and the desired tip speed. In some embodiments, the HSD has a fixed clearance (shear gap width) between the stator and rotor. Alternatively, the HSD has adjustable clearance (shear gap width). The shear gap may be in the range of from about 5 micrometers (0.0002 inch) and about 4 mm (0.016 inch).

Tip speed is the circumferential distance traveled by the tip of the rotor per unit of time. Tip speed is thus a function of the rotor diameter and the rotational frequency. Tip speed (in meters per minute, for example) may be calculated by multiplying the circumferential distance transcribed by the rotor tip, 2πR, where R is the radius of the rotor (meters, for example) times the frequency of revolution (for example revolutions per minute, rpm). The frequency of revolution may be greater than 250 rpm, greater than 500 rpm, greater than 1000 rpm, greater than 5000 rpm, greater than 7500 rpm, greater than 10,000 rpm, greater than 13,000 rpm, or greater than 15,000 rpm. The rotational frequency, flow rate, and temperature may be adjusted to get a desired product profile. If channeling should occur, and some reactants pass through unreacted, the rotational frequency may be increased to minimize undesirable channeling. Alternatively or additionally, unreacted reactants may be introduced into a second or subsequent HSD 40, or a portion of the unreacted reactants may be separated from the products and recycled to HSD 40.

HSD 40 may have a tip speed in excess of 22.9 m/s (4500 ft/min) and may exceed 40 m/s (7900 ft/min), 50 m/s (9800 ft/min), 100 m/s (19,600 ft/min), 150 m/s (29,500 ft/min), 200 m/s (39,300 ft/min), or even 225 m/s (44,300 ft/min) or greater in certain applications. For the purpose of this disclosure, the term ‘high shear’ refers to mechanical rotor stator devices (e.g., colloid mills or rotor-stator dispersers) that are capable of tip speeds in excess of 5.1 m/s. (1000 ft/min) and require an external mechanically driven power device to drive energy into the stream of products to be reacted. By contacting the reactants with the rotating members, which can be made from, coated with, or impregnated with stationary catalyst, significant energy is transferred to the reaction. Especially in instances where the reactants are gaseous, the energy consumption of the HSD 40 will be very low. The temperature may be adjusted to control the product profile and to extend catalyst life.

In some embodiments, HSD 40 is capable of delivering at least 300 L/h at a tip speed of at least 22.9 m/s (4500 ft/min). The power consumption may be about 1.5 kW. HSD 40 combines high tip speed with a very small shear gap to produce significant shear on the material being processed. The amount of shear will be dependent on the viscosity of the fluid in HSD 40. Accordingly, a local region of elevated pressure and temperature is created at the tip of the rotor during operation of HSD 40. In some cases the locally elevated pressure is about 1034.2 MPa (150,000 psi). In some cases the locally elevated temperature is about 500° C. In some cases, these local pressure and temperature elevations may persist for nano or pico seconds.

An approximation of energy input into the fluid (kW/L/min) can be estimated by measuring the motor energy (kW) and fluid output (L/min). As mentioned above, tip speed is the velocity (ft/min or m/s) associated with the end of the one or more revolving elements that is creating the mechanical force applied to the fluid. In embodiments, the energy expenditure of HSD 40 is greater than 1000 watts per cubic meter of fluid therein. In embodiments, the energy expenditure of HSD 40 is in the range of from about 3000 W/m³ to about 7500 kW/m³. In embodiments, the energy expenditure of HSD 40 is in the range of from about 3000 W/m³ to about 7500 W/m³. The actual energy input needed is a function of what reactions are occurring within the HSD, for example, endothermic and/or exothermic reaction(s), as well as the mechanical energy required for dispersing and mixing feedstock materials. In some applications, the presence of exothermic reaction(s) occurring within the HSD mitigates some or substantially all of the reaction energy needed from the motor input. When dispersing a gas in a liquid, the energy requirements are significantly less.

The shear rate is the tip speed divided by the shear gap width (minimal clearance between the rotor and stator). The shear rate generated in HSD 40 may be in the greater than 20,000 s⁻¹. In some embodiments the shear rate is at least 40,000 s⁻¹. In some embodiments the shear rate is at least 100,000 s⁻¹. In some embodiments the shear rate is at least 500,000 s⁻¹. In some embodiments the shear rate is at least 1,000,000 s⁻¹. In some embodiments the shear rate is at least 1,600,000 s⁻¹. In some embodiments the shear rate is at least 3,000,000 s⁻¹. In some embodiments the shear rate is at least 5,000,000 s⁻¹. In some embodiments the shear rate is at least 7,000,000 s⁻¹. In some embodiments the shear rate is at least 9,000,000 s⁻¹. In embodiments where the rotor has a larger diameter, the shear rate may exceed about 9,000,000 s⁻¹. In embodiments, the shear rate generated by HSD 40 is in the range of from 20,000 s⁻¹ to 10,000,000 s⁻¹. For example, in one application the rotor tip speed is about 40 m/s (7900 ft/min) and the shear gap width is 0.0254 mm (0.001 inch), producing a shear rate of 1,600,000 s⁻¹. In another application the rotor tip speed is about 22.9 m/s (4500 ft/min) and the shear gap width is 0.0254 mm (0.001 inch), producing a shear rate of about 901,600 s⁻¹.

In some embodiments, HSD 40 comprises a colloid mill. Suitable colloidal mills are manufactured by IKA® Works, Inc. Wilmington, N.C. and APV North America, Inc. Wilmington, Mass., for example. In some instances, HSD 40 comprises the DISPAX REACTOR® of IKA® Works, Inc.

In some embodiments, each stage of the external HSD has interchangeable mixing tools, offering flexibility. For example, the DR 2000/4 DISPAX REACTOR® of IKA® Works, Inc. Wilmington, N.C. and APV North America, Inc. Wilmington, Mass., comprises a three stage dispersing module. This module may comprise up to three rotor/stator combinations (generators), with choice of fine, medium, coarse, and super-fine for each stage. This allows for variance of shear rate along the direction of flow. In some embodiments, each of the stages is operated with super-fine generator. In some embodiments, at least one of the generator sets has a rotor/stator minimum clearance (shear gap width) of greater than about 5 mm (0.2 inch). In some embodiments, at least one of the generator sets has a rotor/stator minimum clearance (shear gap width) of about 0.2 mm (0.008 inch). In alternative embodiments, at least one of the generator sets has a minimum rotor/stator clearance of greater than about 1.7 mm (0.07 inch).

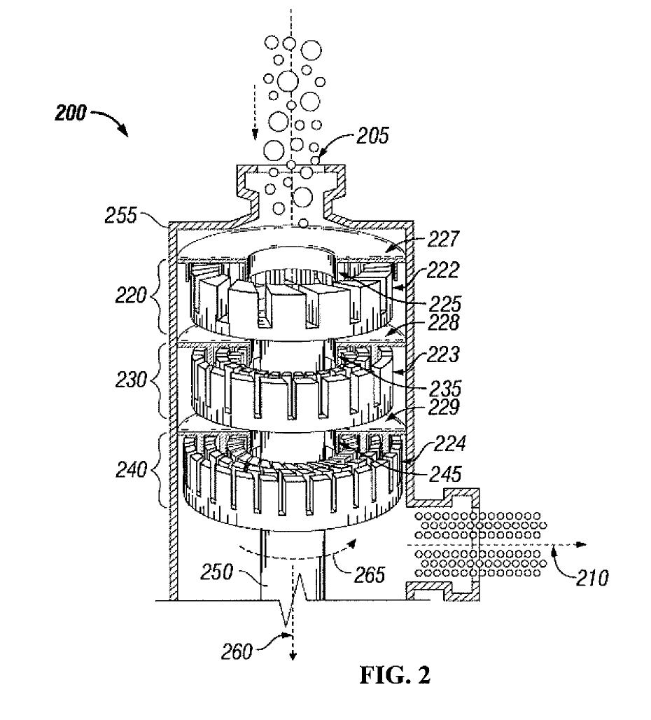

In embodiments, a scaled-up version of the DISPAX REACTOR® is utilized. For example, in embodiments HSD 40 comprises a SUPER DISPAX REACTOR® DRS 2000. The HSD unit may be a DR 2000/50 unit, having a flow capacity of 125,000 liters per hour, or a DRS 2000/50 having a flow capacity of 40,000 liters/hour. Because residence time is increased in the DRS unit, the fluid therein is subjected to more shear. Referring now to FIG. 2, there is presented a longitudinal cross-section of a suitable HSD 200. HSD 200 of FIG. 2 is a dispersing device comprising three stages or rotor-stator combinations, 220, 230, and 240. The rotor-stator combinations may be known as generators 220, 230, 240 or stages without limitation. Three rotor/stator sets or generators 220, 230, and 240 are aligned in series along drive shaft 250.

First generator 220 comprises rotor 222 and stator 227. Second generator 230 comprises rotor 223, and stator 228. Third generator 240 comprises rotor 224 and stator 229. For each generator the rotor is rotatably driven by input 250 and rotates about axis 260 as indicated by arrow 265. The direction of rotation may be opposite that shown by arrow 265 (e.g., clockwise or counterclockwise about axis of rotation 260). Stators 227, 228, and 229 may be fixably coupled to the wall 255 of HSD 200. As mentioned hereinabove, each rotor and stator may comprise rings of complementarily-shaped tips, leading to several shear gaps within each generator.

As discussed above, a contact surface of the HSD 40 may be made from, coated with, or impregnated by a suitable catalyst which catalyzes the desired reaction. In embodiments, a contact surface of one ring of each rotor or stator is made from, coated with, or impregnated with a different catalyst than the contact surface of another ring of the rotor or stator. Alternatively or additionally, a contact surface of one ring of the stator may be made from coated with or impregnated by a different catalyst than the complementary ring on the rotor. The contact surface may be at least a portion of the rotor, at least a portion of the stator, or both. The contact surface may comprise, for example, at least a portion of the outer surface of a rotor, at least a portion of the inner surface of a stator, or at least a portion of both.

As mentioned hereinabove, each generator has a shear gap width which is the minimum distance between the rotor and the stator. In the embodiment of FIG. 2, first generator 220 comprises a first shear gap 225; second generator 230 comprises a second shear gap 235; and third generator 240 comprises a third shear gap 245. In embodiments, shear gaps 225, 235, 245 have widths in the range of from about 0.025 mm to about 10 mm. Alternatively, the process comprises utilization of an HSD 200 wherein the gaps 225, 235, 245 have a width in the range of from about 0.5 mm to about 2.5 mm. In certain instances the shear gap width is maintained at about 1.5 mm. Alternatively, the width of shear gaps 225, 235, 245 are different for generators 220, 230, 240. In certain instances, the width of shear gap 225 of first generator 220 is greater than the width of shear gap 235 of second generator 230, which is in turn greater than the width of shear gap 245 of third generator 240. As mentioned above, the generators of each stage may be interchangeable, offering flexibility. HSD 200 may be configured so that the shear rate remains the same or increases or decreases stepwise longitudinally along the direction of the flow 260.

Generators 220, 230, and 240 may comprise a coarse, medium, fine, and super-fine characterization, having different numbers of complementary rings or stages on the rotors and complementary stators. Although generally less desirable, rotors 222, 223, and 224 and stators 227, 228, and 229 may be toothed designs. Each generator may comprise two or more sets of complementary rotor-stator rings. In embodiments, rotors 222, 223, and 224 comprise more than 3 sets of complementary rotor/stator rings. In embodiments, the rotor and the stator comprise no teeth, thus forcing the reactants to flow through the pores of a sintered material.

HSD 40 may be a large or small scale device. In embodiments, HSD 40 is used to process from less than 10 tons per hour to 50 tons per hour. In embodiments, HSD 40 processes 10 tons/h, 20 tons/h, 30 ton/hr, 40 tons/h, 50 tons/h, or more than 50 tons/h. Large scale units may produce 1000 gal/h (24 barrels/h). The inner diameter of the rotor may be any size suitable for a desired application. In embodiments, the inner diameter of the rotor is from about 12 cm (4 inch) to about 40 cm (15 inch). In embodiments, the diameter of the rotor is about 6 cm (2.4 inch). In embodiments, the outer diameter of the stator is about 15 cm (5.9 inch). In embodiments, the diameter of the stator is about 6.4 cm (2.5 inch). In some embodiments the rotors are 60 cm (2.4 inch) and the stators are 6.4 cm (2.5 inch) in diameter, providing a clearance of about 4 mm. In certain embodiments, each of three stages is operated with a super-fine generator comprising a number of sets of complementary rotor/stator rings.

HSD 200 is configured for receiving at inlet 205 a fluid mixture from line 13. The mixture comprises reactants. The reactants comprise RRG. In embodiments, at least one reactant is gaseous and at least one reactant is liquid. Feed stream entering inlet 205 is pumped serially through generators 220, 230, and then 240, such that product is formed. Product exits HSD 200 via outlet 210 (and line 19 of FIG. 1). The rotors 222, 223, 224 of each generator rotate at high speed relative to the fixed stators 227, 228, 229, providing a high shear rate. The rotation of the rotors pumps fluid, such as the feed stream entering inlet 205, outwardly through the shear gaps (and, if present, through the spaces between the rotor teeth and the spaces between the stator teeth), creating a localized high shear condition. High shear forces exerted on fluid in shear gaps 225, 235, and 245 (and, when present, in the gaps between the rotor teeth and the stator teeth) through which fluid flows process the fluid and create product. The product may comprise a dispersion of unreacted or product gas in a continuous phase of liquid (e.g., liquid product and carrier/catalyst). Product exits HSD 200 via high shear outlet 210 (and line 19 of FIG. 1).

As mentioned above, in certain instances, HSD 200 comprises a DISPAX REACTOR® of IKA® Works, Inc. Wilmington, N.C. and APV North America, Inc. Wilmington, Mass. Several models are available having various inlet/outlet connections, horsepower, tip speeds, output rpm, and flow rate. Selection of the HSD will depend on throughput selection and desired particle, droplet or bubble size in dispersion in line 10 (FIG. 1) exiting outlet 210 of HSD 200. IKA® model DR 2000/4, for example, comprises a belt drive, 4M generator, PTFE sealing ring, inlet flange 25.4 mm (1 inch) sanitary clamp, outlet flange 19 mm (¾ inch) sanitary clamp, 2HP power, output speed of 7900 rpm, flow capacity (water) approximately 300-700 L/h (depending on generator), a tip speed of from 9.4-41 m/s (1850 ft/min to 8070 ft/min). Scale up may be performed by using a plurality of HSDs, or by utilizing larger HSDs. Scale-up using larger models is readily performed, and results from larger HSD 40 units may provide improved efficiency in some instances relative to the efficiency of lab-scale devices. The large scale unit may be a DISPAX® 2000/unit. For example, the DRS 2000/5 unit has an inlet size of 51 mm (2 inches) and an outlet of 38 mm (1.5 inches).

In embodiments wherein strong acid is utilized as carrier, HSD 40 and other portions of system 100 may be made from refractory/corrosion resistant materials. For example, Inconel® alloys, tungsten or Hastelloy® materials may be used.

Vessel. Vessel or reactor 10 is any type of vessel in which a multiphase reaction can be propagated to carry out the above-described conversion reaction(s). For instance, a continuous or semi-continuous stirred tank reactor, or one or more batch reactors may be employed in series or in parallel. In some applications vessel 10 may be a tower reactor, and in others a tubular reactor or multi-tubular reactor. A catalyst inlet line may be connected to vessel 10 for receiving a catalyst solution or slurry during operation of the system. In embodiments where a significant reaction occurs in HSD 40, vessel 10 may comprise one or more fractionators suitable for separating components selected from unreacted and light gas, liquid carrier, catalyst, and value-added product. Vessel 10 may comprise outlet lines for unreacted or light product gas 16, oxygenate product 17 and carrier fluid 20. In embodiments, system 100 comprises distinct apparatus configured to separate unreacted and/or light gas from value-added product, to separate carrier fluid from value-added product, to separate catalyst from value-added product or to separate some combination thereof.

Vessel 10 may include one or more of the following components: stirring system, heating and/or cooling capabilities, pressure measurement instrumentation, temperature measurement instrumentation, one or more injection points, and level regulator, as are known in the art of reaction vessel design. For example, a stirring system may include a motor driven mixer. A heating and/or cooling apparatus may comprise, for example, a heat exchanger. Alternatively, as much of the reaction may occur within HSD 40 in some embodiments, vessel 10 may serve primarily as a storage vessel in some cases. Although generally less desired, in some applications vessel 10 may be omitted, particularly if multiple high shears/reactors are employed in series, as further described below.

RRG Production Apparatus. Dispersible refinery-related gas or RRG may be any suitable refinery-related gas comprising at least one of C1-C8 compounds. The RRG typically comprises C1 to C4 fractions and hydrogen. For example, RRG may comprise any combination of methane, ethane, propane, butane, ethylene, propylene, butylene, carbon monoxide, and carbon dioxide. RRG may also comprise hydrogen and/or sulfur compounds, such as hydrogen sulfide. Most desirably, RRG comprises negative value gas from a refinery. As used herein, negative value gas is a gas whose disposal has a cost and/or is not profitable, such as a gas normally flared or treated in an expensive manner, perhaps prior to flaring. In embodiments, gases used as RRG are those gases typically conventionally used as boiler fuel or flared. In embodiments, RRG comprises gas conventionally introduced into a gas plant of a refinery. In embodiments, RRG comprises pyrolysis gas, coker offgas, FCC offgas, light FCC offgas, associated gas, or a combination thereof.

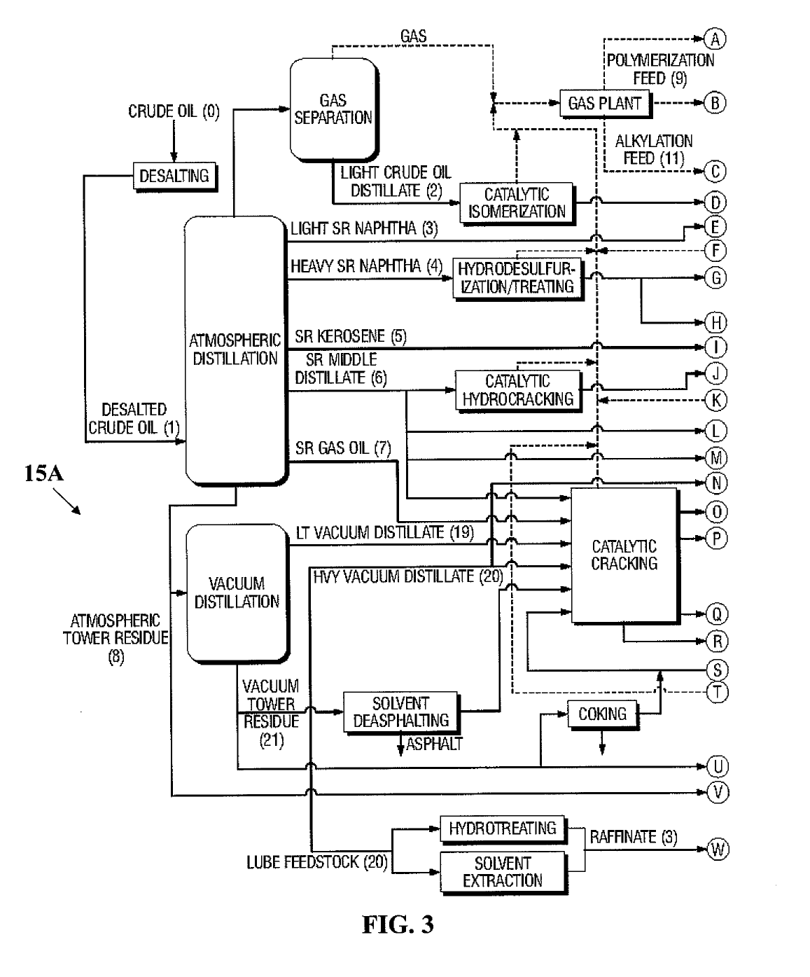

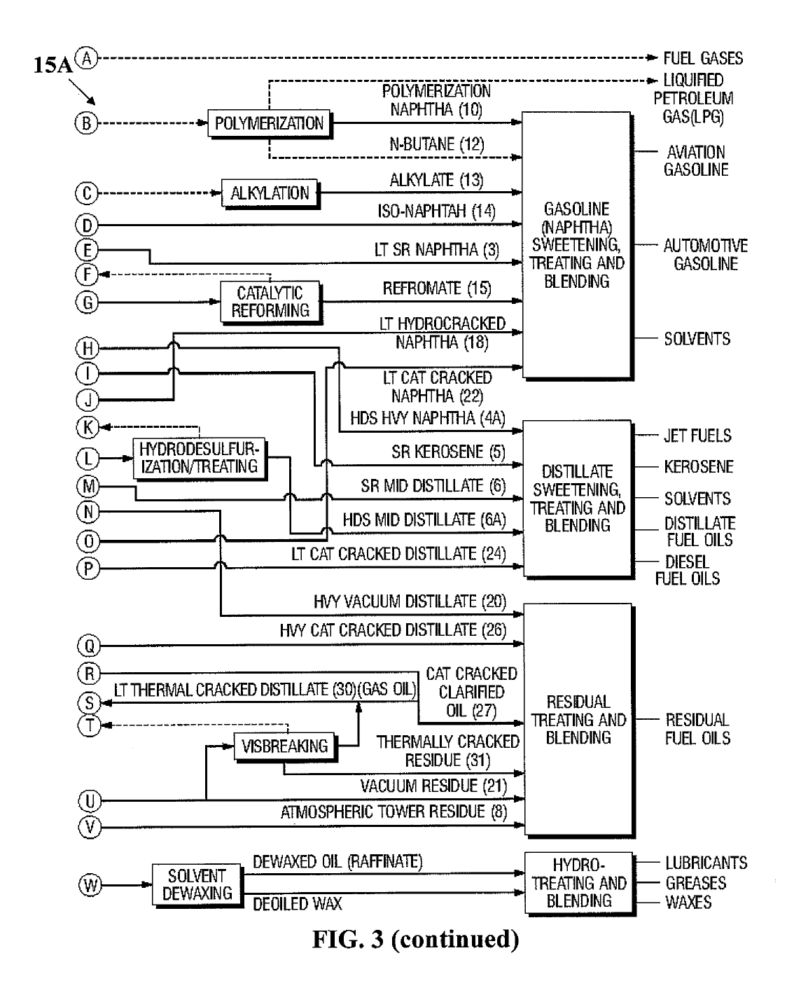

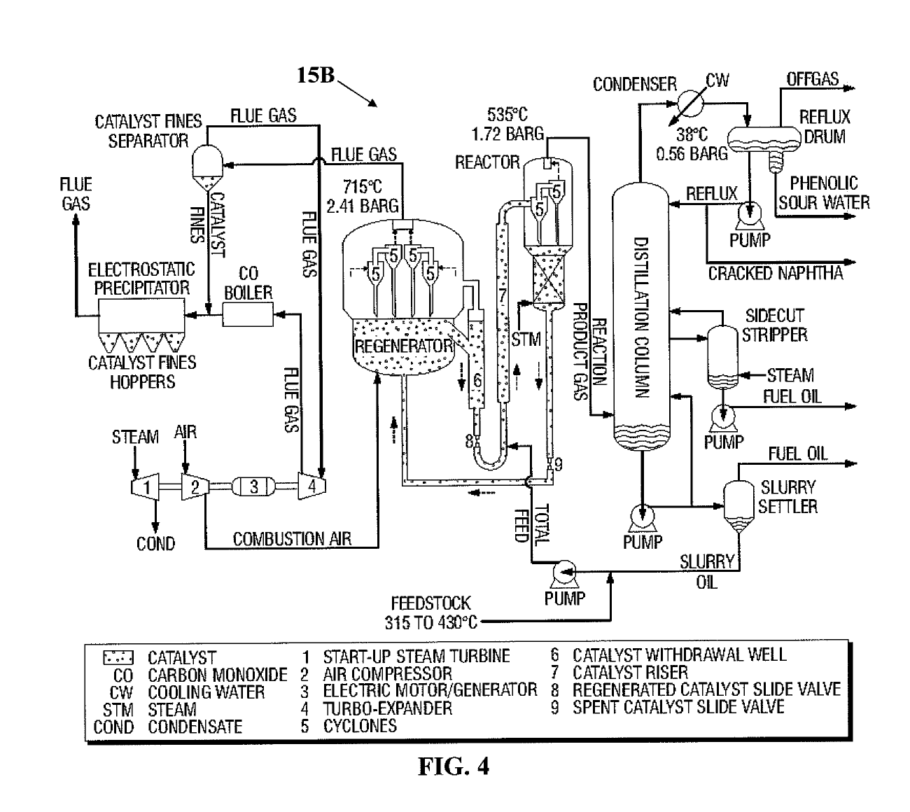

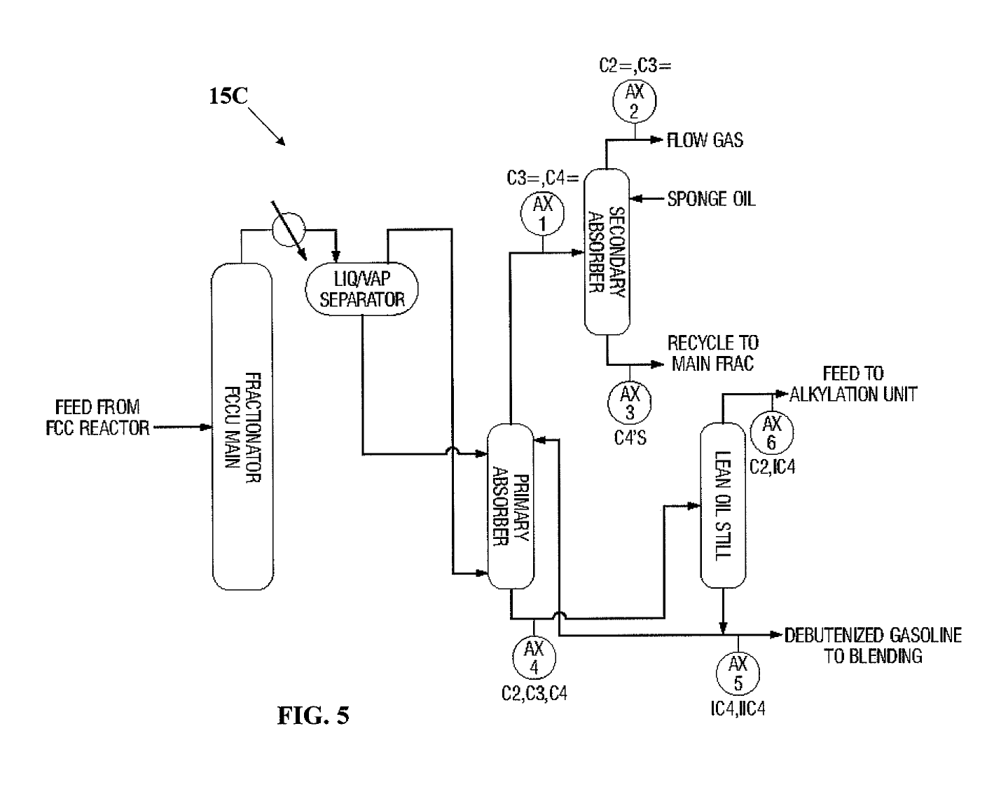

FIG. 3 is a schematic of a typical refinery 15A. RRG production apparatus 15 may be equipment as shown in refinery 15A, or any combination or subset thereof, including multiple of the units indicated. Such equipment and processes are described, for example, in OSHA Technical Manual TED 01-00-015; Section IV, Chapter 2. In embodiments, RRG is derived from or comprises any gas shown directed to the gas plant in FIG. 3. In embodiments, RRG is separated from a crude oil (i.e., as associated gas). In embodiments, RRG is derived from or comprises a gas produced during cracking. In embodiments, RRG is derived from or comprises a gas produced during thermal cracking. For example, in embodiments, RRG is derived from or comprises coker offgas produced by thermal cracking in a coking operation. In embodiments, RRG is derived from or comprises a gas produced during catalytic cracking. In embodiments, RRG is derived from or comprises a gas produced during fluid catalytic cracking. In embodiments, RRG comprises light FCC offgas. RRG production apparatus 15 may be catalytic cracking apparatus known in the art from which an offgas is obtained. In embodiments, RRG production apparatus 15 is any FCC apparatus known in the art for fluid catalytic cracking. For example, FIG. 4 is a schematic of a suitable RRG production apparatus 15B. Some portion of apparatus 15B may be used to provide RRG. In the embodiment of 15B, RRG production apparatus 15B is a FCC system from which an offgas is produced. In embodiments, one or more component of the offgas indicated in FIG. 4 is removed and the remaining gas is utilized as RRG. Any FCC vapor recovery unit known in the art may be used as RRG production apparatus. For example, the offgas of FCC system 15B in FIG. 4 may be fractionated via a system or a portion of the system similar to that of 15C in FIG. 5 prior to use as RRG.

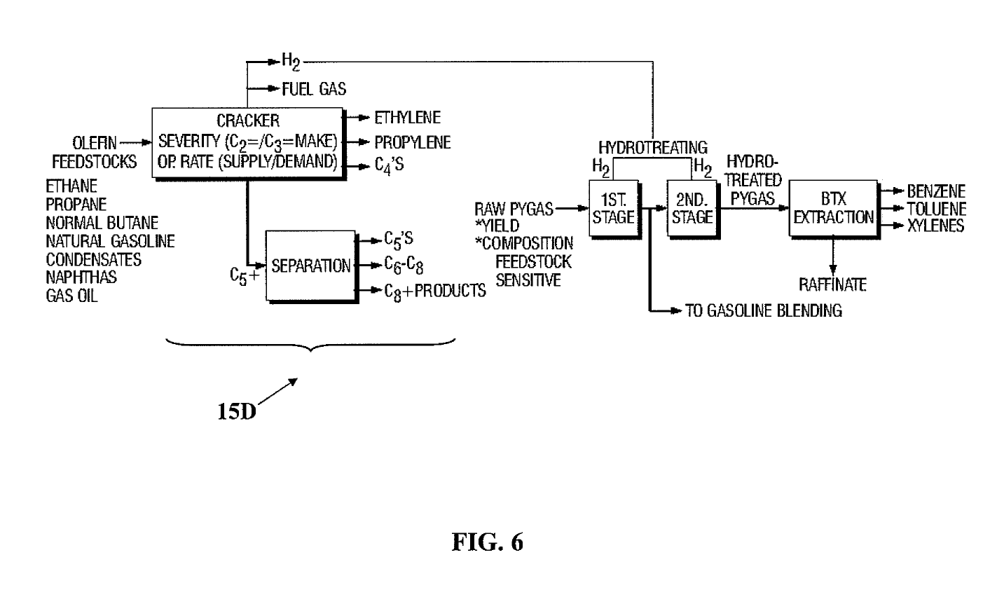

In embodiments, RRG production apparatus 15 comprises steam cracking apparatus from which pyrolysis gas is obtained. Various suitable steam cracking apparatus are known in the art. FIG. 6 is a schematic of a suitable apparatus 15D (i.e. the equipment upstream of the hydrotreating and BTX extraction stages of FIG. 6) for producing pyrolysis gas. In embodiments, RRG production apparatus comprises a steam cracker and may further comprise a separator, as indicated in FIG. 6. In such instances, C6+ gas from a steam cracker, or C6 fraction, C6-C8 or C8+ fraction from a separator may be used as RRG.

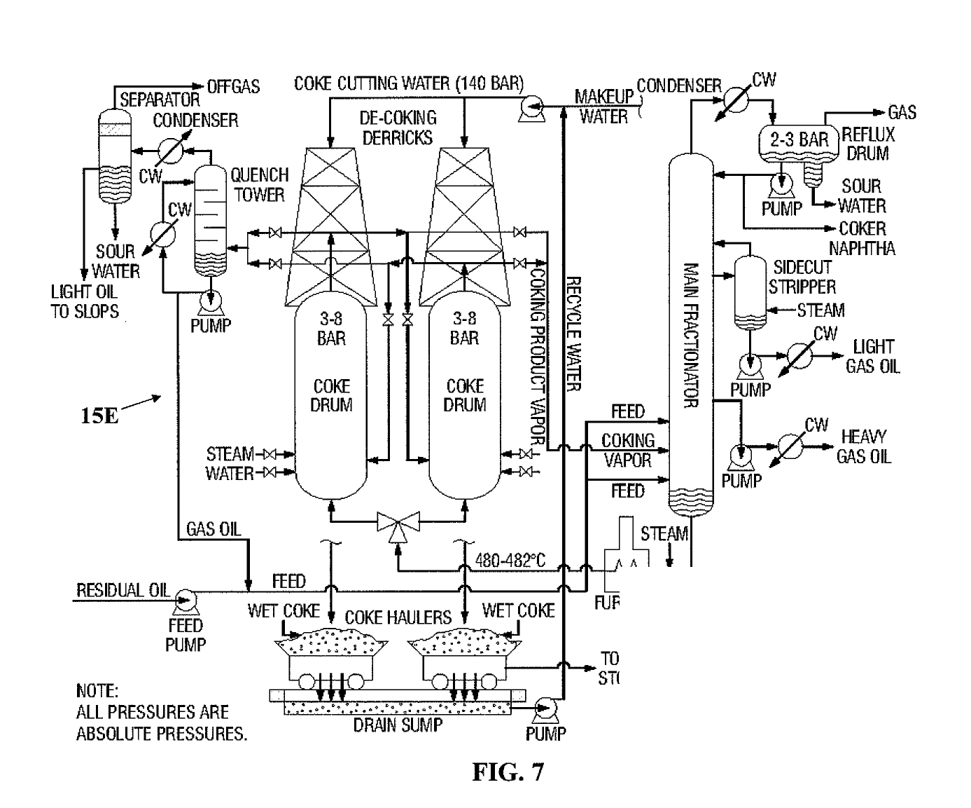

In embodiments, RRG production apparatus 15 comprises coking apparatus, from which coker offgas is obtained/derived. Various suitable coking apparatus are known in the art. For example, a RRG production apparatus 15E as indicated in FIG. 7 or some portion thereof may be utilized in high shear system 100.

Heat Transfer Devices. Internal or external heat transfer devices for heating the fluid to be treated are also contemplated in variations of the system. For example, the reactants may be preheated via any method known to one skilled in the art. Some suitable locations for one or more such heat transfer devices are between pump 5 and HSD 40, between HSD 40 and flow line 19, and between flow line 19 and pump 5 when fluid in flow line 19 is recycled to HSD 40. HSD 40 may comprise an inner shaft which may be cooled, for example water-cooled, to partially or completely control the temperature within HSD 40. Some non-limiting examples of such heat transfer devices are shell, tube, plate, and coil heat exchangers, as are known in the art.

Pumps. Pump 5 is configured for either continuous or semi-continuous operation, and may be any suitable pumping device that is capable of providing controlled flow through HSD 40 and system 100. In applications pump 5 provides greater than 202.65 kPa (2 atm) pressure or greater than 303.97 kPa (3 atm) pressure. Pump 5 may be a Roper Type 1 gear pump, Roper Pump Company (Commerce Georgia) Dayton Pressure Booster Pump Model 2P372E, Dayton Electric Co (Niles, Ill.) is one suitable pump. Preferably, all contact parts of the pump comprise stainless steel, for example, 316 stainless steel. In some embodiments of the system, pump 5 is capable of pressures greater than about 2026.5 kPa (20 atm). In addition to pump 5, one or more additional, high pressure pumps may be included in the system illustrated in FIG. 1. For example, a booster pump, which may be similar to pump 5, may be included between HSD 40 and flow line 19 for boosting the pressure into flow line 19.

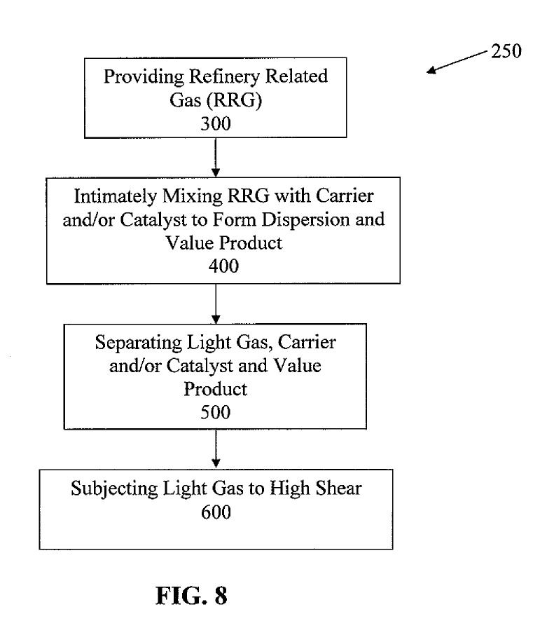

High Shear Process for Producing Value-Added Products from Refinery-Related Gas. A process for producing value-added products from refinery-related gas will be described with respect to FIG. 8 which is a flow diagram of a high shear process 250 according to this disclosure. Process 250 comprises providing refinery-related gas 300; intimately mixing RRG with carrier and/or catalyst at high shear to form a dispersion and a product comprising value-added product 400; and separating unreacted gas, carrier and/or catalyst and value-added product 500. Process 250 may further comprise subjecting light gas to high shear 600.

Providing Refinery-Related Gas, RRG. Providing a refinery-related gas (RRG) 300 may comprise obtaining or producing any refinery-related gas that is conventionally sent to a gas plant. Alternatively or additionally, providing a refinery-related gas may comprise obtaining an associated gas. The modern petroleum refinery utilized for petroleum refining, also called oil refining, utilizes a series of core processes and process units that provide clean gasoline and low sulfur diesel fuel. Various offgases are produced in the refining process. The RRG may comprise pyrolysis gas, FCC offgas, coker offgas, associated gas or any combination thereof Preferably, the RRG is a ‘negative-value gas’ which is conventionally either flared or, when not suitable for flaring, converted at expense to a less undesirable product. The RRG may comprise olefins. Desirably, RRG comprises at least one C1 to C8 compound, hydrogen, or some combination thereof. In embodiments, RRG comprises at least one selected from C1 through C4 compounds and hydrogen.

The RRG may comprise a blast furnace gas having the following or a similar composition: 40-50% (e.g. ˜46%) N₂, 20-25% (e.g. ˜24%) CO, 20-30% (e.g. ˜26%) CO₂, 1-5% (e.g. 4%) H₂, and minor amounts (e.g. less than ˜4%) of O₂ and/or CH₄. The hydrocarbon types in FCC feed are broadly classified as paraffin's, olefins, naphthenes and aromatics (PONA). A description of gases that might conventionally be used as fuel or flared in a refinery operation, and thus suitable for use as RRG according to this disclosure, is provided in Petroleum Refinery Distillation second edition by R. N. Watkins (ISBN 0-87201-672-2), which is hereby incorporated herein in its entirety for all purposes not contradictory to this disclosure.

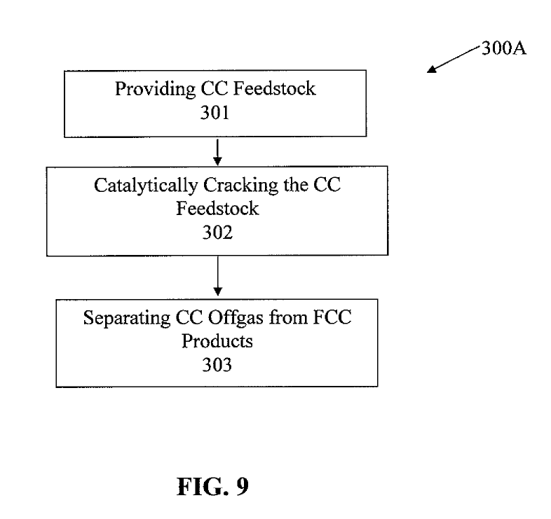

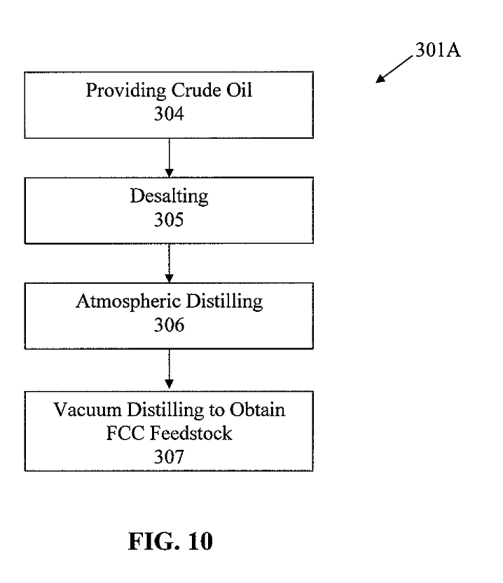

An embodiment for providing refinery-related gas is depicted in the flow diagram of FIG. 9, which is a flow diagram of a method of providing refinery-related gas 300A, wherein the RRG comprises offgas from catalytic cracking (CC). It is noted, however, that the RRG may comprise offgas from thermal (i.e., not catalytic) cracking Catalytic cracking is the process of breaking up heavier hydrocarbon molecules into lighter hydrocarbon fractions by use of heat and catalysts. The method of providing RRG 300A in FIG. 9 comprises providing CC feedstock 301, catalytically cracking the CC feedstock 302, and separating CC offgas from CC products 303. FIG. 10 is a schematic of a suitable process 301A for providing CC feedstock. The CC feedstock may comprise atmospheric gas oil, AGO, and/or vacuum gas oil, VGO. Method 301A for providing CC feedstock comprises providing crude oil 304, desalting the crude oil 305, distilling the desalted crude oil at atmospheric pressure 306, and distilling the atmospheric tower residue from 306 under vacuum to obtain CC feedstock 307.

Providing crude oil 304 comprises providing one or more crude oils via methods known in the art. Crude oils are naturally occurring complex mixtures of hydrocarbons that typically include small quantities of sulfur, nitrogen, and oxygen derivatives of hydrocarbons as well as trace metals. Crude oils contain many different hydrocarbon compounds that vary in appearance and composition from one oil field to another. Crude oils range in consistency from water to tar-like solids, and in color from clear to black. An ‘average’ crude oil contains about 84% carbon, 14% hydrogen, 1%-3% sulfur, and less than 1% each of nitrogen, oxygen, metals, and salts. Crude oils are generally classified as paraffinic, naphthenic, or aromatic, based on the predominant proportion of similar hydrocarbon molecules. Mixed-base crudes contain varying amounts of each type of hydrocarbon. Refinery crude base stocks usually contain mixtures of two or more different crude oils.

Relatively simple crude oil assays are used to classify crude oils as paraffinic, naphthenic, aromatic, or mixed. One assay method (United States Bureau of Mines) is based on distillation, and another method (UOP “K” factor) is based on gravity and boiling points. More comprehensive crude assays may be utilized to estimate the value of the crude (i.e., yield and quality of useful products) and processing parameters. Crude oils are typically grouped according to yield structure.

Crude oils are also defined in terms of API (American Petroleum Institute) gravity. API gravity is an arbitrary scale expressing the density of petroleum products. The higher the API gravity, the lighter the crude. For example, light crude oils have high API gravities and low specific gravities. Crude oils with low carbon, high hydrogen, and high API gravity are usually rich in paraffins and tend to yield greater proportions of gasoline and light petroleum products, while those with high carbon, low hydrogen, and low API gravities are usually rich in aromatics.

Crude oils that contain appreciable quantities of hydrogen sulfide or other reactive sulfur compounds are referred to as ‘sour.’ Crude oils containing less sulfur are referred to as ‘sweet.’ A notable exceptions to this rule are West Texas crude oils, which are always considered ‘sour’ regardless of their hydrogen sulfide content, and Arabian high-sulfur crudes, which are not considered ‘sour’ because the sulfur compounds therein are not highly reactive. Providing crude oil 304 may comprise providing one or more selected from sweet crude oils, sour crude oils, low API crude oils, high API crude oils, medium API crude oils, paraffinic crude oils, naphthenic crude oils, aromatic crude oils, mixed crude oils, or any combination thereof. Table 1 shows typical characteristics, properties, and gasoline potential of various crude oils.

TABLE 1
Typical Approximate Characteristics, Properties
and Gasoline Potential of Various Crude Oils*

| | | | | | | Napht. | |
|---|---|---|---|---|---|---|---|
| | Paraffins | Aromatics | Naphthenes | Sulfur | ~API | Yield | Octane # |
| Source | (vol %) | (vol %) | (vol %) | (wt %) | Gravity | (vol %) | (est.) |
| Nigerian- | 37 | 9 | 54 | 0.2 | 36 | 28 | 60 |
| Light | | | | | | | |
| Saudi- | 63 | 19 | 18 | 2 | 34 | 22 | 40 |
| Light | | | | | | | |
| Saudi- | 60 | 15 | 25 | 2.1 | 28 | 23 | 35 |
| Heavy | | | | | | | |
| Venezuela- | 35 | 12 | 53 | 2.3 | 30 | 2 | 60 |
| Heavy | | | | | | | |
| Venezuela- | 52 | 14 | 34 | 1.5 | 24 | 18 | 50 |
| Light | | | | | | | |
| USA-Midcont. | — | — | — | 0.4 | 40 | — | — |
| Sweet | | | | | | | |
| USA- W. | 46 | 22 | 32 | 1.9 | 32 | 33 | 55 |
| Texas Sour | | | | | | | |
| North Sea- | 50 | 16 | 34 | 0.4 | 37 | 31 | 50 |
| Brent | | | | | | | |

*(representative average values)

Providing CC feedstock may comprise desalting the provided crude. Desalting may be performed as known in the art to remove salt, water and other contaminants from the crude oil prior to distillation in one or more atmospheric tower. Providing CC feedstock may further comprise one or more steps of distilling the crude oil. Crude oil fractionation (distillation) is the separation of crude oil in atmospheric and vacuum distillation towers into groups of hydrocarbon compounds of differing boiling-point ranges called ‘fractions’ or ‘cuts.’ Fractionation separates the crude oil into various fractions or straight-run cuts by distillation in atmospheric and vacuum towers. The main fractions or ‘cuts’ obtained have specific boiling-point ranges and can be classified in order of decreasing volatility into gases, light distillates, middle distillates, gas oils, and residuum.

In embodiments, the desalted crude oil is atmospherically distilled and the atmospheric tower residue obtained during atmospheric distillation is subsequently vacuum distilled. Atmospheric distilling 306 may comprise operating an atmospheric distillation tower as known in the art to fractionate the desalted crude. Atmospheric distilling may comprise separating the crude oil into fractions including naphtha fraction(s), kerosene fraction, diesel fraction, middle distillate fraction, gas oil fraction and a bottoms liquid called atmospheric resid or atmospheric tower residue.

The desalted crude feedstock can be preheated using recovered process heat. The feedstock can be introduced into a direct-fired crude charge heater where it is fed into a vertical distillation column just above the bottom, at pressures slightly above atmospheric and at temperatures ranging from 650° F. to 700° F. (heating crude oil above these temperatures may cause undesirable thermal cracking). All but the heaviest fractions flash into vapor. As the hot vapor rises in the tower, its temperature is reduced. Heavy fuel oil or asphalt residue is taken from the bottom. At successively higher points on the tower, the various major products including lubricating oil, heating oil, kerosene, gasoline, and uncondensed gases (which condense at lower temperatures) may be drawn off.

The fractionating tower used for atmospheric distillation can be any distillation column known in the art. The fractionating tower may comprise a steel cylinder about 120 feet high, containing horizontal steel trays for separating and collecting the liquids. At each tray, vapors from below enter perforations and bubble caps. The perforation and bubble caps permit the vapors to bubble through the liquid on the tray, causing some condensation at the temperature of that tray. An overflow pipe can serve to drain the condensed liquids from each tray back to the tray below, where the higher temperature causes re-evaporation. The evaporation, condensing, and scrubbing operation is repeated many times until the desired degree of product purity is reached. Side streams from certain trays are then taken off to obtain the desired fractions. Products ranging from uncondensed fixed gases at the top to heavy fuel oils at the bottom can be continuously extracted from a fractionating tower. Steam may be used in towers to lower the vapor pressure and create a partial vacuum. The distillation process separates the major constituents of crude oil into so-called straight-run products. Sometimes crude oil is ‘topped’ by distilling off only the lighter fractions, leaving a heavy residue that is often distilled further under high vacuum.

Distilling under vacuum 307 may be performed by any method known in the art. In embodiments, vacuum distilling 307 comprises fractionating the atmospheric tower residue via vacuum distillation into gas oil, light vacuum distillate, heavy vacuum distillate, vacuum resid, or a combination thereof. Vacuum distillation is the distillation of petroleum under vacuum which reduces the boiling temperature sufficiently to prevent cracking or decomposition of the feedstock. In embodiments, the CC feedstock comprises gas oil from atmospheric and/or vacuum distilling, light vacuum distillate, heavy vacuum distillate, or a combination thereof.

In vacuum distilling, in order to further distill the residuum or topped crude from the atmospheric distillation tower at higher temperatures, reduced pressure is utilized to prevent thermal cracking Vacuum distilling may be performed in one or more vacuum distillation towers. The principles of vacuum distillation resemble those of fractional distillation and the equipment is also similar, except that larger-diameter columns may be used to maintain comparable vapor velocities at the reduced pressures. The internal designs of the vacuum tower may be different from the atmospheric distillation tower in that random packing and demister pads may be used instead of trays. A typical first-phase vacuum tower may be used to produce gas oils, lubricating-oil base stocks, and heavy residual for propane deasphalting. Deasphalting is a process of removing asphaltic materials from reduced crude using liquid propane to dissolve nonasphaltic compounds. A second-phase tower operating at lower vacuum may be used to distill surplus residuum from the atmospheric tower, which is not used for lube-stock processing, and surplus residuum from the first vacuum tower not used for deasphalting. One or more vacuum tower can be used to separate the catalytic cracking feedstock from surplus residuum.

Providing RRG may further comprise subjecting the CC feedstock to catalytic cracking 302. Catalytic cracking breaks complex hydrocarbons into simpler molecules in order to increase the quality and quantity of lighter, more desirable products and decrease the amount of residuals. This process rearranges the molecular structure of hydrocarbon compounds to convert heavy hydrocarbon feedstock into lighter fractions such as kerosene, gasoline, LPG, heating oil, and petrochemical feedstock.

Catalytic cracking is similar to thermal cracking except that catalysts facilitate the conversion of the heavier molecules into lighter products. Use of a catalyst (i.e., a material that assists a chemical reaction but does not take part in it) in the cracking reaction increases the yield of improved-quality products under much less severe operating conditions than in thermal cracking. Typical temperatures are from 850° F. to 950° F. at much lower pressures of 10 to 20 psi. The catalysts used in the cracking unit may be solid materials (e.g., zeolite, aluminum hydrosilicate, treated bentonite clay, fuller's earth, bauxite, and silica-alumina) that come in the form of powders, beads, pellets or are shaped materials called extrudites. In catalytic cracking, catalytic cracking feedstock reacts with catalyst and cracks into different hydrocarbons; catalyst is reactivated by burning off coke; and the cracked hydrocarbon products are separated into various products.

The RRG can be obtained or derived via any of the three types of catalytic cracking processes: fluid catalytic cracking (FCC), moving-bed catalytic cracking, and Thermofor catalytic cracking (TCC). The catalytic cracking process is very flexible, and operating parameters can be adjusted to meet changing product demand. The offgas composition utilized as RRG may vary depending on operating parameters of the cracking. In addition to cracking, catalytic activities include dehydrogenation, hydrogenation, and/or isomerization. Table 2 indicates the feedstock and typical products of catalytic cracking processes. As indicated, all or a portion of the off gas from catalytic cracking may, according to this disclosure, be utilized as RRG.

In embodiments, providing RRG comprises providing offgas from a fluid catalytic cracker, FFC, for example a FCC as shown in FIG. 4. Fluid catalytic cracking (FCC) is the most important conversion process used in petroleum refineries. It is widely used to convert the high-boiling hydrocarbon fractions of petroleum crude oils to more valuable gasoline, olefinic gases (light olefins) and other product. The FCC feedstock may comprise a fraction of the crude oil that has an initial boiling point of 340° C. or higher at atmospheric pressure and an average molecular weight ranging from about 200 to 600 or higher. The FCC process vaporizes and breaks the long-chain molecules of the high-boiling hydrocarbon liquids into much shorter molecules by contacting the feedstock, at high temperature and moderate pressure, with a fluidized powdered catalyst. Subjecting CC feedstock to catalytic cracking 302 may comprise cracking the oil feedstock (i.e., the FCC feedstock) in the presence of a finely divided catalyst, by any means known in the art. The FCC catalyst may be maintained in an aerated or fluidized state by the oil vapors. The fluid catalytic cracker may contain a catalyst section and a fractionating section that operate together as an integrated processing unit. The catalyst section can contain the reactor and regenerator, which, with the standpipe and riser, can form the catalyst circulation unit. The fluid catalyst can be continuously circulated between the FCC reactor and the regenerator using air, oil vapors, and/or steam as the conveying media.

TABLE 2
Catalytic Cracking Process

| | | | Typical | |
|---|---|---|---|---|
| Feedstock | From | Process | Products | Sent to |
| Gas Oils | Distillation | Decomposition, | Gasoline | Treater or |
| | towers, coker, | alteration | | Blending |
| | visbreaker | | Off Gases | HSD |
| Deasphalted | Deasphalter | | Middle | Hydrotreater, |
| Oils | | | Distillates | blending or |
| | | | | recycle |
| | | | Petrochem | Petrochem |
| | | | Feedstock | or other |
| | | | Residue | Residual |
| | | | | Fuel Blend |

In embodiments, FCC is carried out by mixing a preheated hydrocarbon charge (i.e., the FCC feedstock) with hot, regenerated catalyst as it enters the riser leading to the FCC reactor. The charge is combined with a recycle stream within the riser, vaporized, and raised to reactor temperature (900° to 1,000° F.) by the hot catalyst. As the mixture travels up the riser, the charge is cracked at 10 to 30 psi. In embodiments utilizing modern FCC units, all cracking can occur in the riser. The FCC ‘reactor’ may thus merely serve as a holding vessel for the cyclones. Cracking continues until the oil vapors are separated from the catalyst in the reactor cyclones.

Providing RRG via 300A further comprises separating the CC offgas from the CC products. In embodiments in which the catalytic cracking is fluid catalytic cracking, the resultant FCC product stream (cracked product) may be fractionated into various fractions, including an FCC offgas fraction which is utilized as the provided RRG. The products of the fluid catalytic cracker may thus be introduced into an FCC product fractionating column where it is separated into fractions, including an FCC offgas fraction.

Spent FCC catalyst can be regenerated to eliminate coke that collects on the catalyst during the FCC process. Spent catalyst flows through the catalyst stripper to the regenerator, where most of the coke deposits burn off at the bottom where preheated air and spent catalyst are mixed. Fresh catalyst is added and worn-out catalyst removed to optimize the cracking process.

Utilizing FCC offgas for the RRG of the disclosed method may be more desirable than conventional treatment of such offgas. Conventionally, the main fractionator offgas is sent to what is called a gas recovery unit where it is separated into butanes and butylenes, propane and propylene, and lower molecular weight gases (hydrogen, methane, ethylene and ethane). Some conventional FCC gas recovery units also separate out some of the ethane and ethylene.

Conventionally, olefins recovery from refinery FCC offgas streams has been used to provide cash flow from olefins from a tail-gas stream that has typically been consumed as refinery fuel or flared. Such recovery schemes can be employed in refineries or olefins plants, and can be tailored to fit individual requirements. However, the conventional treatment of FCC off-gas is, complex and capital intensive. In embodiments, as shown in FIG. 4, FCC offgas is further treated, for example, as shown in FIG. 5, to remove olefins as known in the art and only the light gas remaining after removal of various products is utilized as RRG. As shown in FIG. 5, all or a portion of the light C1 to C4 gases that are taken off the FCC unit and may also contain H₂, CO and/or S, may be used as RRG according to this disclosure.

In embodiments, subjecting the CC feedstock to catalytic cracking 302 comprises subjecting the CC feedstock to moving bed catalytic cracking, by methods known in the art. The moving-bed catalytic cracking process is similar to the FCC process. The catalyst is in the form of pellets that are moved continuously to the top of the unit by conveyor or pneumatic lift tubes to a storage hopper, then flow downward by gravity through the reactor, and finally to a regenerator. The regenerator and hopper are isolated from the reactor by steam seals. The cracked product is separated into recycle gas, oil, clarified oil, distillate, naphtha, and wet gas. The gas or wet gas or a portion thereof may be used as provided RRG.

In embodiments, subjecting the CC feedstock to catalytic cracking 302 comprises subjecting the CC feedstock to Thermofor catalytic cracking, as known in the art. In a typical thermofor catalytic cracking unit, the preheated feedstock flows by gravity through the catalytic reactor bed. The vapors are separated from the catalyst and sent to a fractionating tower, from which CC offgas may be obtained for use as RRG. The spent catalyst is regenerated, cooled, and recycled. The flue gas from regeneration is sent to a carbon-monoxide boiler for heat recovery.

In embodiments, providing RRG comprises recovering offgas (which is normally sent to the gas plant of a refinery) from thermal cracking. Thermal cracking is the breaking up of heavy oil molecules into lighter fractions by the use of high temperature without the aid of catalysts. Thermal cracking subjects heavy fuels to both pressure and intense heat, physically breaking the large molecules into smaller ones to produce additional gasoline and distillate fuels. The thermal cracking utilized to produce the RRG as offgas may be visbreaking, another form of thermal cracking.

Because the simple distillation of crude oil produces amounts and types of products that are not consistent with those required by the marketplace, subsequent refinery processes change the product mix by altering the molecular structure of the hydrocarbons. One of the ways of accomplishing this change is through ‘cracking,’ a process that breaks or cracks the heavier, higher boiling-point petroleum fractions into more valuable products such as gasoline, fuel oil, and gas oils. The two basic types of cracking are thermal cracking, using heat and pressure, and catalytic cracking, which is discussed above.

The RRG may be an offgas (conventionally sent to gas plant, fuel, or flare) of a thermal cracking process selected from visbreaking, steam cracking, coking, and combinations thereof.

The RRG may be obtained via visbreaking Visbreaking is a mild form (low temperature) of thermal cracking that significantly lowers the viscosity or pour point of heavy crude-oil residue (straight-run residuum) without affecting the boiling point range. Residual from an atmospheric distillation tower may be heated (800° F. to 950° F.) at atmospheric pressure and mildly cracked in a heater. It may then be quenched with cool gas oil to control overcracking, and flashed in a distillation tower. Visbreaking is conventionally used to reduce the pour point of waxy residues and reduce the viscosity of residues used for blending with lighter fuel oils. Middle distillates may also be produced via visbreaking, depending on product demand. The thermally cracked residue tar, which accumulates in the bottom of the fractionation tower, is vacuum flashed in a stripper and the distillate recycled. Table 3 indicates typical feedstocks and resulting products of visbreaking, and indicates the potential use of the offgas of visbreaking operations or a portion thereof for RRG.

In embodiments, providing RRG 300 comprises providing pyrolysis gas. Pyrolysis gas is a by-product from the manufacture of ethylene by steam cracking of hydrocarbon fractions such as naphtha or gas oil. Pyrolysis gasoline or pygas may be obtained or produced as a byproduct in a steam cracking olefin plant and may consist of C5- to C10-hydrocarbons. A suitable pyrolysis gas production apparatus is indicated in FIG. 6. Pygas is generally used as a feedstock for the production of aromatics (e.g. benzene), but is also sometimes applied for other purposes such as gasoline production. Because the raw pygas contains unstable or undesired components such as dienes, olefins and sulfur components, the stream is conventionally subjected to (2-stage) hydrogenation or hydrotreatment. Hydrotreating also can be employed to improve the quality of pyrolysis gasoline (pygas), a by-product from the manufacture of ethylene. Traditionally, the outlet for pygas has been motor gasoline blending, a suitable route in view of its high octane number. However, only small portions can be blended untreated owing to the unacceptable odor, color, and gum-forming tendencies of this material. The quality of pygas, which is high in diolefin content, is conventionally sometimes improved by hydrotreating, whereby conversion of diolefins into mono-olefins provides an acceptable product for motor gas blending. Such hydrotreatment may be undesirable in light of the method of producing oxygenates presented herein, which may utilize pygas to produce valuable products.

TABLE 3
Visbreaking Process

| | | | Typical | |
|---|---|---|---|---|
| Feedstock | From | Process | Products | To |
| Residual | Atmospheric | Decompose | Gasoline or | Hydrotreating |
| | Tower and | | Distillate | |
| | Vacuum | | Vapor | Hydrotreating |
| | Tower | | Residue | Stripper or |
| | | | | Recycle |
| | | | Offgas | HSD |

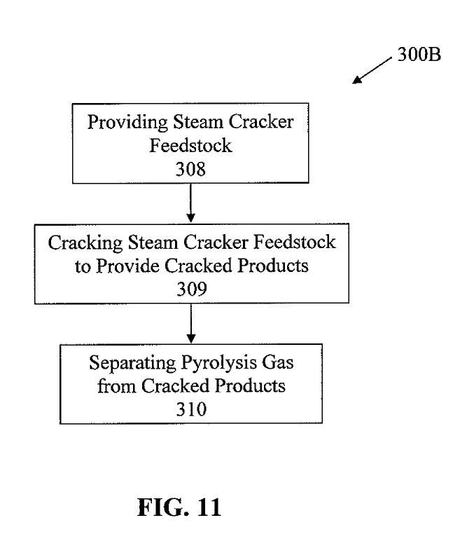

According to embodiments of this disclosure, pygas may be utilized for the production of value-added products. RRG may be provided via the method of providing RRG 300B presented in the flowchart of FIG. 11. Providing RRG 300B comprises providing steam cracker feedstock 308, cracking the steam cracker feedstock to provide cracked products 309, and separating pyrolysis gas from cracked products 310. Providing steam cracker feedstock 308 may comprise producing or obtaining any suitable steam cracker feedstock as known in the art. The steam cracker feedstock comprises naphtha. Naphtha is a general term used for low boiling hydrocarbon fractions that are a major component of gasoline. Aliphatic naphtha refers to those naphthas containing less than 0.1% benzene and with carbon numbers from C3 through C16. Aromatic naphthas have carbon numbers from C6 through C16 and contain significant quantities of aromatic hydrocarbons such as benzene (>0.1%), toluene, and xylene. Naphtha is used primarily as feedstock for producing a high octane gasoline component (via the catalytic reforming process). It is also used in the petrochemical industry for producing olefins in steam crackers and in the chemical industry for solvent (cleaning) applications.

The steam cracker feedstock may range from ethane to vacuum gas oil, with heavier feeds giving higher yields of by-products such as naphtha. The steam cracker feedstock may comprise ethane, butane, naphtha, or a combination thereof. Cracking steam cracker feedstock to provide cracked products 309 comprises introducing the steam cracker feedstock into a steam cracker, which is a petrochemical apparatus that converts a steam cracker feedstock (e.g. naphtha and perhaps light hydrocarbons) into olefins (e.g. ethylene, propylene), and other chemical raw materials. In embodiments, the steam cracking is carried out at temperatures of 1,500° F. to 1,600° F., and at pressures slightly above atmospheric. Following cracking of the steam cracker feedstock 309, the pyrolysis gas is separated from the cracked products at 310. The cracked products (chemicals) can be processed as conventionally, e.g. transported, via pipeline and other methods, to petrochemical and polymer facilities and converted into olefin-based products. Naphtha produced from steam cracking typically contains benzene, which is extracted prior to hydrotreating. Residual from steam cracking is sometimes blended into heavy fuels. The pyrolysis gas may be provided as RRG 300 according to this disclosure.

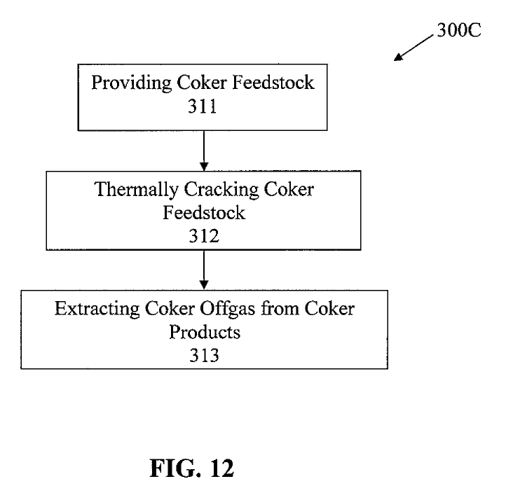

In embodiments, providing RRG 300 comprises producing or obtaining coker offgas by any method known in the art. An exemplary system for providing coker offgas is provided in FIG. 7. FIG. 12 is a flow diagram of a method of producing coker offgas for providing as RRG 300C according to an embodiment of this disclosure. Providing RRG 300C comprises providing coker feedstock 311, thermally cracking coker feedstock 312, and extracting coker offgas from coker products 313. The coker offgas may be obtained from coking, which is a process for thermally converting and upgrading heavy residual into lighter products and by-product petroleum coke. Coke is the high carbon-content residue remaining from the destructive distillation of petroleum residue.

Coking is a severe method of thermal cracking used to upgrade heavy residuals into lighter products or distillates. Providing coker feedstock 311 may comprise providing residual from an atmospheric tower and/or a vacuum distillation tower. Coking of coker feedstock may produce straight-run gasoline (coker naphtha) and various middle-distillate fractions used as catalytic cracking feedstock, along with coker offgas for use as RRG according to this disclosure. Coking so completely reduces hydrogen that the residue is a form of carbon called ‘coke.’ Delayed coking and/or continuous (contact or fluid) coking may provide the coker offgas for use as RRG.

In embodiments, RRG is provided as offgas from delayed coking Vacuum resid is conventionally processed in delayed coking units which convert heavy oil from crude into lighter products. In delayed coking the heated charge (coker feedstock, typically residuum from atmospheric distillation tower) is transferred to large coke drums which provide the long residence time needed to allow the cracking reactions to proceed to completion. Initially the heavy feedstock is fed to a furnace which heats the residuum to high temperatures (900° F. to 950° F.) at low pressures (25 to 30 psi) and is designed and controlled to prevent premature coking in the heater tubes. The mixture is passed from the heater to one or more coker drums where the hot material is held approximately 24 hours (delayed) at pressures of 25 to 75 psi, until it cracks into lighter products. Vapors from the drums are returned to a fractionator where offgas, naphtha, and gas oils are separated. The coker offgas may be used to provide RRG 300.

Conventionally, when the coke reaches a predetermined level in one drum, the flow is diverted to another drum to maintain continuous operation. The full drum is steamed to strip out uncracked hydrocarbons, cooled by water injection, and decoked by mechanical or hydraulic methods. The coke may be mechanically removed by an auger rising from the bottom of the drum. Hydraulic decoking consists of fracturing the coke bed with high-pressure water ejected from a rotating cutter.

In embodiments, RRG is provided as offgas from continuous coking. Continuous (contact or fluid) coking is a moving-bed process that operates at temperatures higher than delayed coking. In continuous coking, thermal cracking occurs by using heat transferred from hot, recycled coke particles to feedstock in a radial mixer, called a reactor, at a pressure of 50 psi. Gases and vapors are taken from the reactor, quenched to stop any further reaction, and fractionated. As indicated in Table 4 which tabulates typical feedstocks and products of coking operations, the coker offgas or a portion thereof may be used as RRG according to this disclosure. The reacted coke enters a surge drum and is lifted to a feeder and classifier where the larger coke particles are removed as product. The remaining coke is dropped into the preheater for recycling with feedstock. Coking occurs both in the reactor and in the surge drum. The process is automatic in that there is a continuous flow of coke and feedstock. As mentioned hereinabove, potential off gas compositions are also described in Petroleum Refinery Distillation second edition by R. N. Watkins (ISBN 0-87201-672-2).

TABLE 4
Coking Processes

| | | | Typical | |
|---|---|---|---|---|
| Feedstock | From | Process | Products | To |
| Residual | Atm./Vacuum | Decomp. | Naphtha/ | Distillation/ |
| | Tower | | gasoline | Blending |
| | Catalytic Cracker | | | |
| Clarified Oil | Catalytic Cracker | | Coke | Shipping/ |
| | | | | Recycle |
| Tars | Various | | Gasoil | Catalytic |
| | | | | Cracking |
| | | | Wastewater | Treatment |
| | | | (sour) | |
| | | | Offgas | HSD |

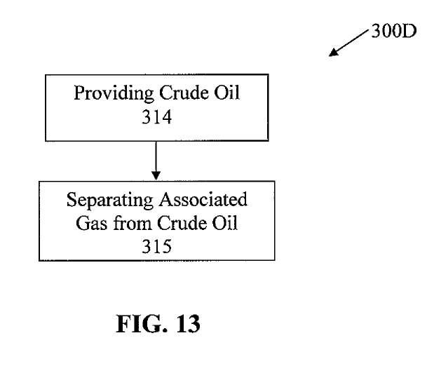

In embodiments, providing RRG 300 comprises providing associated gas. A method of providing associated gas 300D utilizing associated gas is presented in FIG. 13. Providing RRG 300D comprises providing crude oil 314 and separating associated gas from crude oil 315. Providing crude oil 314 may be performed as with step 304 described above in relation to FIG. 10. Associated gas is gas found dissolved in crude oil at the high pressures existing in a reservoir, or gas present as a gas cap over the oil. Associated gas comprises natural gas. Separating associated gas from crude oil may be performed as known in the art.

Other refinery-related gas may be used as RRG according to this disclosure. Any gas conventionally sent to a gas plant can be used as RRG and converted to value-added product via the method of this disclosure. For example, offgas produced during hydrodesulfurization or a portion thereof may be used to provide RRG. Hydrodesulfurization refers to a catalytic process in which the principal purpose is to remove sulfur from petroleum fractions in the presence of hydrogen. In embodiments, one or more product is removed from a gas conventionally sent to a gas treating plant prior to its use as RRG. Unsaturated and or saturated gas plants may remove one or more components prior to use of the gas as RRG. For example, butanes and butenes may be removed for use as alkylation feedstock, heavier components may be sent to gasoline blending, propane may be recovered for LPG, and propylene may be removed for use in petrochemicals.

Intimately Mixing RRG with Carrier and/or Catalyst to Form Value-Added Product. The disclosed process for the production of value-added products from refinery-related gas 250 further comprises intimately mixing the provided RRG with carrier and/or catalyst to form a dispersion and value-added product 400. The value-added products may comprise olefins and/or oxygenates, including alcohols. Incorporating one or more HSD 40 into a conventional refinery may be especially desirable. Sulfuric acid may be a most suitable carrier, as sulfuric acid is the most commonly used acid treating process found in a typical oil refinery. Additionally, the RRG will typically comprise sulfur, and the high shear process may convert the sulfur in the RRG to sulfuric acid, which may be removed with the carrier. Sulfuric acid treatment is a process in which unfinished petroleum products such as gasoline, kerosene, and lubricating oil stocks are treated with sulfuric acid to improve color, odor, and other properties.

Conventional sulfuric acid treating results in partial or complete removal of unsaturated hydrocarbons, sulfur, nitrogen, and oxygen compounds, and resinous and asphaltic compounds. It is used to improve the odor, color, stability, carbon residue, and other properties of the oil. A portion of the sulfuric acid at the refinery may be used to produce value-added product from various RRGs according to this disclosure.

Intimately mixing 400 may comprise subjecting a mixture of the RRG and carrier and/or catalyst to a shear rate of at least 20,000 s⁻¹ or greater, as further discussed hereinbelow. Intimately mixing 400 may comprise mixing to form a dispersion comprising bubbles of RRG dispersed in the carrier (which may be or contain catalyst), wherein the bubbles have an average particle diameter of about 5, 4, 3, 2, 1, or less than 1 micron. In embodiments, the bubbles have an average particle diameter in the nanometer range, the micron range, or the submicron range.

Referring now to FIG. 1, intimately mixing 400 may comprise introducing a suitable RRG via dispersible gas stream 22 and a carrier and/or catalyst via stream 21 into a high shear device 40. The HSD may be a rotor-stator device as described hereinabove.

In operation, a dispersible gas stream comprising RRG is introduced into system 100 via line 22, and combined in line 13 with a carrier stream to form a gas-liquid stream. The carrier may be or contain therein a catalyst. The carrier 21 may be any suitable liquid carrier, and may be aqueous or organic. In embodiments, the carrier comprises sulfuric acid, which also acts as a catalyst. In embodiments, the carrier and/or catalyst is selected from sulfuric acid, phosphoric acid, sulfonic acid, and combinations thereof. In embodiments, a catalyst suitable for catalyzing a hydration reaction is employed. An inert gas such as nitrogen may be used to fill reactor 10 and purge it of any air and/or oxygen prior to operation of system 100. According to an embodiment, the catalyst is phosphoric acid disposed on a solid support such as without limitation, silica. In other embodiments, the catalyst may be sulfuric acid or sulfonic acid. In embodiments, the catalyst comprises a zeolite. Examples of the zeolites usable in various embodiments include crystalline aluminosilicates such as mordenite, erionite, ferrierite and ZSM zeolites developed by Mobil Oil Corp.; aluminometallosilicates containing foreign elements such as boron, iron, gallium, titanium, copper, silver, etc.; and metallosilicates substantially free of aluminum, such as gallosilicates and borosilicates. As regards the cationic species which are exchangeable in the zeolites, the proton-exchanged type (H-type) zeolites are usually used, but it is also possible to use the zeolites which have been ion-exchanged with at least one cationic species, for example, an alkaline earth element such as Mg, Ca and Sr, a rare earth element such as La and Ce, a VIII-group element such as Fe, Co, Ni, Ru, Pd and Pt, or other element such as Ti, Zr, Hf, Cr, Mo, W and Th. Catalyst may be fed into reactor 10 through a catalyst feed stream. Alternatively, catalyst may be present in a fixed or fluidized bed 10.

Alternatively, the dispersible gas may be fed directly into HSD 40, instead of being combined with the carrier (e.g. sulfuric acid) in line 13. Pump 5 is operated to pump the carrier through line 21, and to build pressure and feed HSD 40, providing a controlled flow throughout high shear (HSD) 40 and high shear system 100. In some embodiments, pump 5 increases the pressure of the HSD inlet stream in line 13 to greater than 200 kPa (2 atm) or greater than about 300 kPa (3 atmospheres). In this way, high shear system 100 may combine high shear with pressure to enhance intimate mixing of reactant(s).

In a preferred embodiment, dispersible RRG gas may continuously be fed into the carrier stream 13 to form the high shear feed stream (e.g. a gas-liquid feed stream). In high shear device 40, carrier and the RRG are highly dispersed such that nanobubbles and/or microbubbles of RRG are formed for superior dissolution of RRG into solution. Once dispersed, the dispersion may exit high shear device 40 at high shear outlet line 19. Stream 19 may optionally enter vessel 10. Vessel 10 may comprise a fluidized or fixed bed and be used in lieu of or in addition to a slurry catalyst process. However, (e.g. in a slurry catalyst embodiment), high shear outlet stream 19 may directly enter reactor/vessel 10 for further reaction. The reaction stream may be maintained at the specified reaction temperature, using cooling coils in the reactor 10 to maintain reaction temperature. Reaction products (e.g. value-added product which may comprise olefins, alcohols, and/or other oxygenates) may be withdrawn at product stream 17. Unreacted/light gas may be removed from vessel 10 via line 16. Carrier may be recycled via line 20. Vessel 10 may include one or more separation vessels for the separation of any combination of value-added products, light gas, carrier liquid, and catalyst.

Because the RRG will vary depending on the source of the RRG, the reactions occurring in HSD 40 and/or vessel 10 and the resulting value-added product will vary. Reactions that may occur are FT reactions (e.g. when RRG comprises carbon monoxide and hydrogen, i.e., synthesis gas; FT catalyst may be utilized), olefin hydration reactions (e.g. when RRG comprises olefins; carrier may comprise sulfuric acid; zeolite catalyst may be present), methanol production (e.g. when RRG comprises methane; carrier may comprise sulfuric acid), cracking reactions, and various other reactions, as known in the art and discernible without undue reaction, via experimentation with a desired RRG. As mentioned, FT reactions may occur within system 100. Such reactions are described in U.S. patent application Ser. No. 12/138,269, which is hereby incorporated herein in its entirety for all purposes not inconsistent with this disclosure. Olefin hydration reactions may occur in system 100. Such reactions are described in U.S. Pat. No. 7,482,497 and U.S. patent application Ser. Nos. 12/335,270 and 12/140,763, each of which is incorporated hereby herein in its entirety for all purposes not inconsistent with this disclosure.

Value-added product will generally comprise at least one component selected from oxygenates and olefins. In embodiments, value-added product comprises at least one alcohol. The alcohol may comprise ethanol, propanol, isopropanol, butanol, or a combination thereof. High shear conversion of olefin feedstock to product comprising alcohol is described in U.S. patent application Ser. No. 12/335,270, which is hereby incorporated herein in its entirety for all purposes consistent with this disclosure. Any source of OH can be used to form the alcohol, for example water may provide the OH source. In embodiments, for example, RRG comprises FCC offgas. In embodiments, the FCC offgas comprises ethylene and/or ethane. In such and/or other embodiments, the value-added product comprises primarily alcohols. In embodiments, the value-added product comprises at least one selected from oxygenates. In embodiments, the value-added product comprises at least one selected from alcohols.

The reactants are intimately mixed within HSD 40, which serves to subject the reactants to high shear. It is also envisaged that a catalyst may additionally be present in the reactant stream in certain embodiments. For example, a solid, gaseous or liquid phase catalyst may be introduced to HSD 40 via inlet line 13, line 21, or line 22. In an exemplary embodiment, the high shear device comprises a commercial disperser such as IKA® model DR 2000/4, a high shear, three stage dispersing device configured with three rotors in combination with stators, aligned in series, as described above. The disperser is used to create the dispersion of RRG in the liquid carrier. The rotor/stator sets may be configured as illustrated in FIG. 2, for example. In such an embodiment, the combined reactants enter the high shear device via line 13 and enter a first stage rotor/stator combination having circumferentially spaced first stage shear openings. The coarse dispersion exiting the first stage enters the second rotor/stator stage, which has second stage shear openings. The reduced bubble-size dispersion emerging from the second stage enters the third stage rotor/stator combination having third stage shear openings. The rotors and stators of the generators may have circumferentially spaced complementarily-shaped rings. The dispersion exits the high shear device via line 19. In some embodiments, the shear rate increases stepwise longitudinally along the direction of the flow 260, or going from an inner set of rings of one generator to an outer set of rings of the same generator. In other embodiments, the shear rate decreases stepwise longitudinally along the direction of the flow, 260, or going from an inner set of rings of one generator to an outer set of rings of the same generator (outward from axis 200). For example, in some embodiments, the shear rate in the first rotor/stator stage is greater than the shear rate in subsequent stage(s). For example, in some embodiments, the shear rate in the first rotor/stator stage is greater than or less than the shear rate in a subsequent stage(s). In other embodiments, the shear rate is substantially constant along the direction of the flow, with the stage or stages being the same. If HSD 40 includes a PTFE seal, for example, the seal may be cooled using any suitable technique that is known in the art. The HSD 40 may comprise a shaft in the center which may be used to control the temperature within HSD 40. For example, the carrier stream flowing in line 13 may be used to cool the seal and in so doing be preheated as desired prior to entering the high shear device. Heat may be added to HSD 40 (via the shaft or elsewhere, such as external to HSD 40) to promote reactions, in embodiments.

The rotor(s) of HSD 40 may be set to rotate at a speed commensurate with the diameter of the rotor and the desired tip speed. As described above, the HSD (e.g., colloid mill or toothed rim disperser) has either a fixed clearance between the stator and rotor or has adjustable clearance.

HSD 40 serves to intimately mix the RRG and the carrier. In some embodiments of the process, the transport resistance of the reactants is reduced by operation of the high shear device such that the velocity of the one or more reaction (i.e. reaction rate) is increased by greater than a factor of about 5. In some embodiments, the velocity of the reaction is increased by at least a factor of 10. In some embodiments, the velocity is increased by a factor in the range of about 10 to about 100 fold. In some embodiments, HSD 40 delivers at least 300 L/h at a nominal tip speed of at least 22 m/s (4500 ft/min), 40 m/s (7900 ft/min), and which may exceed 225 m/s (45,000 ft/min) or greater. The power consumption may be about 1.5 kW or higher as desired. Although measurement of instantaneous temperature and pressure at the tip of a rotating shear unit or revolving element in HSD 40 is difficult, it is estimated that the localized temperature seen by the intimately mixed reactants may be in excess of 500° C. and at pressures in excess of 500 kg/cm² under high shear conditions.

The rate of chemical reactions involving liquids, gases and solids depend on time of contact, temperature, and pressure. In cases where it is desirable to react two or more raw materials of different phases (e.g. solid and liquid; liquid and gas; solid, liquid and gas), one of the limiting factors controlling the rate of reaction involves the contact time of the reactants. When reaction rates are accelerated, residence times may be decreased, thereby increasing obtainable throughput.

In the case of heterogeneously catalyzed reactions there is the additional rate limiting factor of having the reacted products removed from the surface of the solid catalyst to permit the catalyst to catalyze further reactants. Contact time for the reactants and/or catalyst is often controlled by mixing which provides contact with reactants involved in a chemical reaction.

Not to be limited by theory, it is known in emulsion chemistry that sub-micron particles, or bubbles, dispersed in a liquid undergo movement primarily through Brownian motion effects. Such sub-micron sized particles or bubbles may have greater mobility through boundary layers of solid catalyst particles, thereby facilitating and accelerating the catalytic reaction through enhanced transport of reactants.

The high shear results in dispersion of the RRG in micron or submicron-sized bubbles or droplets. In some embodiments, the resultant dispersion has an average bubble size less than about 1.5 μm. Accordingly, the dispersion exiting HSD 40 via line 19 comprises micron and/or submicron-sized gas bubbles. In some embodiments, the mean bubble size is in the range of about 0.4 μm to about 1.5 μm. In some embodiments, the resultant dispersion has an average bubble or droplet size less than or about 1 μm. In some embodiments, the mean bubble size is less than about 400 nm, and may be less than or about 100 nm in some cases. In many embodiments, the microbubble dispersion is able to remain dispersed at atmospheric pressure for at least 15 minutes.

Once dispersed, the resulting dispersion exits HSD 40 via line 19 and feeds into vessel 10, as illustrated in FIG. 1. As a result of the intimate mixing of the reactants prior to entering vessel 10, a significant portion of the chemical reactions may take place in HSD 40, with or without the presence of a catalyst. Accordingly, in some embodiments, reactor/vessel 10 may be used primarily for heating and separation of volatile reaction products from the value-added product. Alternatively, or additionally, vessel 10 may serve as a primary reaction vessel where most of the value-added product is produced. Vessel/reactor 10 may be operated in either continuous or semi-continuous flow mode, or it may be operated in batch mode. The contents of vessel 10 may be maintained at a specified reaction temperature using heating and/or cooling capabilities (e.g., cooling coils) and temperature measurement instrumentation, employing techniques that are known to those of skill in the art. Pressure in the vessel may be monitored using suitable pressure measurement instrumentation, and the level of reactants in the vessel may be controlled using a level regulator, employing techniques that are known to those of skill in the art. The contents are stirred continuously or semi-continuously.

Conditions of temperature, pressure, space velocity and reactant composition may be adjusted to produce a desired product profile. The use of HSD 40 may allow for better interaction and more uniform mixing of the reactants and may therefore permit an increase in possible throughput and/or product yield. In some embodiments, the operating conditions of system 100 comprise a temperature of at or near ambient temperature and global pressure of at or near atmospheric pressure. Because the HSD 40 provides high pressure (e.g. 150,000 psi) at the tips of the rotors, the temperature of the reaction may be reduced relative to conventional reaction systems in the absence of high shear. In embodiments, the operating temperature is less than about 70% of the conventional operating temperature, or less than about 60% of the conventional operating temperature, or less than about 50% of the conventional operating temperature for the same reaction(s)

The residence time within HSD 40 is typically low. For example, the residence time can be in the millisecond range, can be about 10, 20, 30, 40, 50, 60, 70, 80, 90 or about 100 milliseconds, can be about 100, 200, 300, 400, 500, 600, 700, 800, or about 900 milliseconds, can be in the range of seconds, or can be any range thereamong.

Commonly known hydration reaction conditions may suitably be employed as the conditions to promote production of an alcohol by hydrating olefins in RRG by using catalysts. There is no particular restriction as to the reaction conditions. The hydration reaction of an olefin is an equilibrium reaction to the reverse reaction, that is, the dehydration reaction of an alcohol, and a low temperature and a high pressure are ordinarily advantageous for the formation of an alcohol. However, preferred conditions greatly differ according to the particular starting olefin. From the viewpoint of the rate of reaction, a higher temperature is preferred. Accordingly, it is difficult to simply define the reaction conditions. However, in embodiments, a reaction temperature may range from about 50° C. to about 350° C., preferably from about 100° C. to about 300° C. Furthermore, the reaction pressure may range from about 1 to 300 atmospheres, alternatively 1 to 250 atmospheres.

If a catalyst is used to promote the reactions, it may be introduced directly into vessel 10, as an aqueous or nonaqueous slurry or stream. Alternatively, or additionally, catalyst may be added elsewhere in system 100. For example, catalyst slurry may be injected into line 21. In some embodiments, line 21 may contain a flowing fresh carrier stream and/or a recycle stream from vessel 10.

The bulk or global operating temperature of the reactants is desirably maintained below their flash points. In some embodiments, the operating conditions of system 100 comprise a temperature in the range of from about 50° C. to about 300° C. In specific embodiments, the reaction temperature in vessel 10, in particular, is in the range of from about 90° C. to about 220° C. In some embodiments, the reaction pressure in vessel 10 is in the range of from about 5 atm to about 50 atm.

The dispersion may be further processed prior to entering vessel 10, if desired. In vessel 10, reactions (e.g. olefin hydration) continue. The contents of the vessel are stirred continuously or semi-continuously, the temperature of the reactants is controlled (e.g., using a heat exchanger), and the fluid level inside vessel 10 is regulated using standard techniques. Reaction may occur either continuously, semi-continuously or batch wise, as desired for a particular application.

Separating Light Gas, Carrier, Catalyst and Value-Added Product(s). Method 250 further comprises separating light gas, carrier and value-added product 500. In instances where the carrier is not the catalyst or another catalyst (e.g., solid catalyst) is utilized, 500 may further comprise separating the solid catalyst from the other components in vessel 10. This separation may be performed via vessel 10 or via separate separation vessels. Any light reaction gas that is produced or unreacted components of RRG may exit reactor 10 via gas line 16. In embodiments, this gas stream is recycled to HSD 40. Any suitable separation method known in the art may be used to separate the light gas, carrier liquid, catalyst (if present), and value-added products. For example, one or more of vapor liquid separations, solid/liquid separations, distillations, and other separation means may be used to separate the desired components exiting HSD 40 and/or vessel 10.

Subjecting Light Gas to High Shear. Method 250 may further comprise subjecting light gas to high shear 600. The light gas 16 may comprise carbon dioxide, hydrogen, methane, and various other light components. In embodiments, subjecting light gas 16 to high shear 600 comprises intimately mixing light gas in the presence of FT catalyst, whereby FT product is produced. In this manner, gas-to-liquids production of FT liquid hydrocarbons may be effected. Any suitable FT catalyst may be utilized. The high shear FT process may be carried out as described in U.S. patent Ser. No. 12/138,269. In such embodiments, a portion of the HSD may be made from or coated with FT catalyst, slurry of FT catalyst may be circulated, or vessel 10 may comprise a fixed or slurry bed of FT catalyst. Liquid hydrocarbons may be extracted from vessel 10.

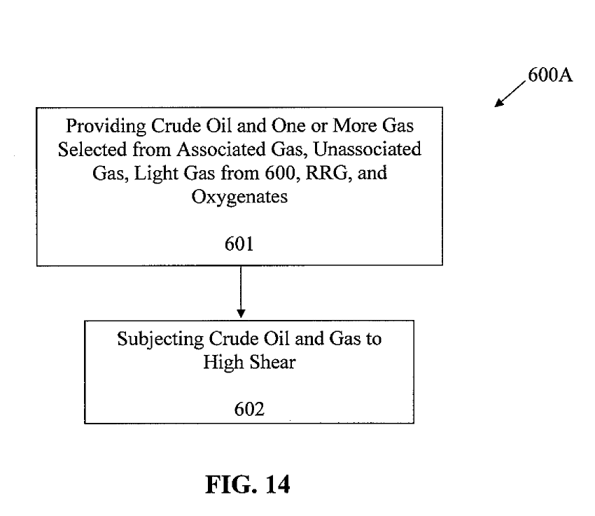

Subjecting light gas to high shear, 600 may comprise intimately mixing the crude oil and the light gas. In an embodiment, as shown in FIG. 14, the gas in line 16 is utilized in a method 600A of stabilizing and/or altering the API gravity of crude oil, as indicated in FIG. 14. This method comprises providing crude oil and gas selected from associated gas, unassociated gas, light gas from step 600 of FIG. 1, RRG, oxygenates and combinations thereof 601 and subjecting the crude oil and the selected gas to high shear 602. RRG may be obtained as described in relation to FIGS. 9-13. Associated gas may be obtained as described in relation to FIG. 13. The phrase ‘unassociated gas’ herein refers to gas obtained in a reservoir in the absence of oil, as known in the art. The crude oil may be provided as described with respect to step 304 in FIG. 10 and step 314 in FIG. 13 hereinabove. The crude oil and selected gas may be subjected to high shear by introduction into a HSD 40, as discussed hereinabove. In embodiments, crude oil extracted from the earth with associated gas is intimately mixed via HSD 40 (desirably before pressure reduction) to adjust the stability and/or the API gravity thereof. In embodiments, crude oil extracted from the earth (with or without associated gas) is intimately mixed with unassociated gas via HSD 40 to adjust the stability/API gravity thereof. Intimately mixing the crude oil with the selected gas may comprise running the crude oil through one or more HSDs 40. Intimately mixing the crude oil with the selected gas may comprise running the crude oil through two or more HSDs 40. Intimately mixing the crude oil with the selected gas may comprise running the crude oil through three or more HSDs 40. Additional selected gas may be introduced into each subsequent HSD. Method 600A may be utilized to alter the API gravity and/or stabilize the crude oil, by reducing volatile components therein. In embodiments, the API is increased by a factor of at least or about 1.5 or 2 by the method of 600A. In embodiments, the API of a crude oil is increased from about 15 to about 30, from about 5 to about 20, or from about 10 to about 20 via method 600A. Method 600A may be utilized to reduce the production of undesirable asphaltenes during refinery operations. The term ‘asphaltenes’ refers to the asphalt compounds soluble in carbon disulfide but insoluble in paraffin naphthas.

The value-added product recovered via line 17 may be further treated as known in the art. For example, value-added product may be contacted with cold water to remove sulfuric acid therefrom. The products, for example oxygenates, may be used and/or separated as known in the art. Separated components may be recycled, as desired.

Carbon Dioxide Reduction. In an embodiment, carbon dioxide (considered as a RRG gas and a greenhouse gas) and water are converted to a value-added product. In some embodiments, the value added-product comprises alcohols such as methanol. In some other embodiments, the value added-product comprises aldehydes and ketones and other organic oxygenates.

In some embodiments, the carbon dioxide source is a Refinery-Related Gas (RRG) from a power plant, which includes mainly N₂, CO₂, water, some O₂, CO, sulfur, and nitrous oxides. In petroleum refining embodiments, where there is little unsaturation, sulfur, and/or oxygen, hydrogen is present. In some embodiments, the carbon dioxide source is a blast furnace gas. In some cases, the main composition of the blast furnace gas is 46% nitrogen, 24% carbon monoxide, 26% carbon dioxide, 4% hydrogen, some amount of oxygen and methane. In some embodiments, the carbon dioxide source is a FCC off-gas. In some cases, the main composition of the FCC off-gas is 1.1% H₂, 13.31% N₂, 1.54% CO, 27.48% CH₄, 22.94% C₂H₄, 23.35% C₂H₆, 2.11% C₃H₆, 0.4% C₃H₈, 4.61% C₄H₈, 0.27% C₄H₁₀, and 2.63% C₅₊.

In some embodiments, the carbon dioxide comes from a fossil fuel (e.g., coal, natural gas, petroleum) burning facility (FFBF) or some components thereof. In some cases, the fossil fuel burning facility (FFBF) is a power plant or a power station. In some other cases, the FFBF is a burner or furnace. Such FFBF's are known to one skilled in the art. This disclosure does not intend to differentiate the FFBF by its size, purpose of function, or mechanism of operation.

In some embodiments, the conversion of carbon dioxide and water is promoted by a bio-catalytic substance (e.g., an enzyme). In some embodiments, the reaction is promoted by electro catalytic methods. In some cases, carbon dioxide and water are converted to methanol.

In some embodiments, the conversion of carbon dioxide and water is promoted by a bio-catalytic substance (e.g., an enzyme) in conjunction with an inorganic catalyst as described hereinabove. In some cases, carbon dioxide and water are converted to alcohols, aldehydes and ketones and other organic oxygenates.

Without wishing to be limited by theory, it is contemplated that the reaction between carbon dioxide and water is accelerated through the creation of free radicals from H₂O and CO₂ under high shear conditions. Furthermore, the intimated mixing and cavitation effects caused by high shear reduce mass transfer limitations so that the reaction rate is increased.

Various dimensions, sizes, quantities, volumes, rates, and other numerical parameters and numbers have been used for purposes of illustration and exemplification of the principles of the invention, and are not intended to limit the invention to the numerical parameters and numbers illustrated, described or otherwise stated herein. Likewise, unless specifically stated, the order of steps is not considered critical. The different teachings of the embodiments discussed below may be employed separately or in any suitable combination to produce desired results.

Multiple Pass Operation. In the embodiment shown in FIG. 1, the system is configured for single pass operation, wherein the product produced in HSD 40 continues along flow line 17. The output of HSD 40 may be run through a subsequent HSD. In some embodiments, it may be desirable to pass the contents of flow line 19, or a fraction thereof, through HSD 40 during a second pass. In this case, at least a portion of the contents of flow line 19 may be recycled from flow line 19 and pumped by pump 5 into line 13 and thence into HSD 40. Additional reactants may be injected via line 22 into line 13, or may be added directly into the HSD. In other embodiments, product is further treated prior to recycle of a portion thereof to HSD 40.

Multiple HSDs. In some embodiments, two or more HSDs like HSD 40, or configured differently, are aligned in series, and are used to promote further reaction. Operation of the mixers may be in either batch or continuous mode. In some instances in which a single pass or “once through” process is desired, the use of multiple HSDs in series may also be advantageous. In embodiments, the reactants pass through multiple HSDs 40 in serial or parallel flow. For example, in embodiments, product in outlet line 19 is fed into a second HSD. When multiple HSDs 40 are operated in series or in parallel, additional reactants and/or carrier (liquid or gaseous) may be injected into the inlet feedstream of each HSD. For example, different dispersible gas, such as hydrogen, carbon dioxide, and/or carbon monoxide may be introduced into a second or subsequent HSD 40. In embodiments, gas comprising oxygenate is injected into the inlet feedstream. For example, gas comprising carbon monoxide, carbon dioxide, oxygen, light alcohols, or a combination thereof may be introduced into the inlet of each in a series or parallel arrangement of HSDs.

For example, a first HSD 40 may be used to convert RRG comprising FCC offgas comprising ethylene and/or ethane to product comprising ethanol and/or other oxygenate(s) and/or higher hydrocarbons. Gas remaining or produced within HSD 40 exits vessel 10 via light product gas outlet line 16. Gas in light product outlet line 16 may be recycled to HSD 40 or introduced into a second HSD along with liquid carrier. The light gas in light gas outlet line 16 may comprise hydrogen, for example. The light gas in light gas outlet line 16 may be introduced into the serial HSD along with additional gas, for example, another available RRG. The additional gas may comprise, for example, carbon dioxide, carbon monoxide, methane, or a combination thereof. For example, carbon monoxide and/or carbon dioxide may be available from regeneration of FCC catalyst. The same or a different catalyst may be used in HSD 40 and a second or subsequent HSD. The catalyst may be selected based upon the gas to be treated therein. In some embodiments, multiple HSDs 40 are operated in parallel, and the outlet products therefrom are introduced into one or more flow lines 19. Any gas remaining following treatment via the disclosed method may be utilized as fuel or flared. This amount will generally be much less than the amount of gas conventionally used for fuel or flare in a typical refinery.

Features. The intimate contacting of reactants within HSD 40 may result in faster and/or more complete reaction of reactants. In embodiments, use of the disclosed process comprising reactant mixing via external HSD 40 allows use of reduced quantities of catalyst than conventional configurations and methods and/or increases the product yield and/or the conversion of reactants. In embodiments, the method comprises incorporating external HSD 40 into an established process thereby reducing the amount of catalyst required to effect desired reaction(s) and/or enabling an increase in production throughput from a process operated without HSD 40, for example, by reducing downtime involved in replacement of catalyst in a conventional slurry bed reactor. In embodiments, the disclosed method reduces operating costs and/or increases production from an existing process. Alternatively, the disclosed method may reduce capital costs for the design of new processes.

Without wishing to be limited to a particular theory, it is believed that the level or degree of high shear mixing may be sufficient to increase rates of mass transfer and also produce localized non-ideal conditions (in terms of thermodynamics) that enable reactions to occur that would not otherwise be expected to occur based on Gibbs free energy predictions. Localized non ideal conditions are believed to occur within the HSD resulting in increased temperatures and pressures with the most significant increase believed to be in localized pressures. The increases in pressure and temperature within the HSD are instantaneous and localized and quickly revert back to bulk or average system conditions once exiting the HSD. Without wishing to be limited by theory, in some cases, the HSD may induce cavitation of sufficient intensity to dissociate one or more of the reactants into free radicals, which may intensify a chemical reaction or allow a reaction to take place at less stringent conditions than might otherwise be required. Cavitation may also increase rates of transport processes by producing local turbulence and liquid micro-circulation (acoustic streaming). An overview of the application of cavitation phenomenon in chemical/physical processing applications is provided by Gogate et al., “Cavitation: A technology on the horizon,” Current Science 91 (No. 1): 35-46 (2006). The HSD of certain embodiments of the present system and methods may induce cavitation whereby one or more reactant is dissociated into free radicals, which then react. In embodiments, the extreme pressure at the tips of the rotors/stators leads to liquid phase reaction, and no cavitation is involved.

### EXAMPLES

The following section provides further details regarding examples of various embodiments.

CO₂ and crude are passed through a high shear unit and water and CO₂ are used to create alcohol (high shear: 1000 rpm; reactor with CO₂ at 90° C. and 100 psig).

Possible mechanism: CO₂ reacts with the ruthenium carbonyl to produce a ruthenium oxide, which then catalyzes the reaction of CO with water to produce the hydrogen and more carbon dioxide. Hydrogen may then react with carbon monoxide to produce methanol (CO+2H₂=CH₃OH), which is possibly catalyzed by the produced Ruthenium oxide.

The analysis of the aqueous phase of the sample by Gas Chromatography (GC) reveals that the aqueous phase of the sample contains approximately 65% methanol.

### Experimental Procedure

| TIME | ACTIVITY DESCRIPTION |
|---|---|
| 10:00 am | Dissolved 5 grams Ru3 in 500 ml of 70 weight oil from |
| | Conoco. Added an additional 3½ L of 70 weight oil to the |
| | reactor. Started 3 heaters and circulation pump. |
| 10:05 | Turn shear pump on. Temperature 23° C. |
| 10:17 | 57° C. at reactor; 0 psig |
| 10:27 | Turn Water injection pump on and started CO2 injection; |
| | reactor temp 70° C. Injected 1 L water through injection |
| | pump. |
| 10:40 | Reactor temp 78° C. Added an additional 500 ml water |
| | through injection pump. |
| 11:50 | Blew all water out of jacket - temp 87° C. - cut one heater off. |
| 10:55 | Temp 86° C. - added 1 L water to injection pump |
| 11:00 | Added 500 ml water to injection pump. Temp 86° C. |
| 11:45 | Cut water injection off. Total of 3 L injected. Temp 82° C. |
| | Reactor at 100 psi. |
| 1:00 pm | Temp 84° C. reactor pressure 100 psig. Terminated |
| | experiment. - Aqueous sample taken and labeled MBM 11 |

### Gas Chromatography (GC) specifications (Model Agilent 6850; Detector: TCD)

| | |
|---|---|
| Column | Model Agilen 19095N-123E |
| | AP-INNOWax Polyethylene Glycol |
| | Capilarity 30.0 m × 530 m × 1 μm nominal |
| Operating conditions | Inlet temperature 175 C. |
| | Carrire gas Helium |
| | Column pressure 3.99 psi |
| | Oven temperature 75 C. hold time 6 min |
| | Flow rate 4.7 ml/min |

While preferred embodiments of the invention have been shown and described, modifications thereof can be made by one skilled in the art without departing from the spirit and teachings of the invention. The embodiments described herein are exemplary only, and are not intended to be limiting. Many variations and modifications of the invention disclosed herein are possible and are within the scope of the invention. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, and so forth). Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, and the like.

Accordingly, the scope of protection is not limited by the description set out above but is only limited by the claims which follow, that scope including all equivalents of the subject matter of the claims. Each and every claim is incorporated into the specification as an embodiment of the present invention. Thus, the claims are a further description and are an addition to the preferred embodiments of the present invention. The disclosures of all patents, patent applications, and publications cited herein are hereby incorporated by reference, to the extent they provide exemplary, procedural or other details supplementary to those set forth herein.

What is claimed is:

1. A method of producing value-added product from refinery-related gas, the method comprising:
(a) providing a refinery-related gas comprising at least one compound selected from the group consisting of C1-C8 compounds and combinations thereof;
(b) intimately mixing the refinery-related gas with a liquid carrier in a high shear device to form a dispersion of gas in the liquid carrier, wherein the gas bubbles in the dispersion have a mean diameter of less than or equal to about 5 micron(s); and
(c) extracting value-added product comprising at least one component selected from the group consisting of higher hydrocarbons, olefins, alcohols, aldehydes, and ketones.

2. The method of claim 1 wherein the refinery-related gas is selected from the group consisting of pyrolysis gas, FCC offgas, associated gas, hydrodesulfurization offgas, coker offgas, catalytic cracker offgas, thermal cracker offgas, and combinations thereof.

3. The method of claim 1 wherein the C1-C8 compounds comprise carbon dioxide.

4. The method of claim 1 wherein the alcohol is selected from the group consisting of methanol, ethanol, isopropanol, butanol, and propanol.

5. The method of claim 1, wherein (b) further comprises contacting the refinery-related gas and the carrier with a catalyst.

6. The method of claim 5, wherein the catalyst comprises at least one component selected from the group consisting of phosphoric acid, sulfonic acid, sulfuric acid, zeolites, solid acid catalysts, and liquid acid catalysts.

7. The method of claim 1 wherein the carrier is a catalyst.

8. The method of claim 7 wherein the carrier comprises sulfuric acid.

9. The method of claim 1 wherein the carrier comprises water.

10. The method of claim 1 wherein (c) comprises separating a light gas from the carrier and the value-added product.

11. The method of claim 1 further comprising contacting the carrier and the refinery-related gas with a catalyst selected from the group consisting of hydrogenation catalysts, hydroxylation catalysts, partial oxidation catalysts, hydrodesulfurization catalysts, hydrodenitrogenation catalysts, hydrofinishing catalysts, reforming catalysts, hydration catalysts, hydrocracking catalysts, Fischer-Tropsch catalysts, dehydrogenation catalysts, and polymerization catalysts.

12. A method of increasing the API gravity of a crude oil, the method comprising:
introducing the crude oil and a gas selected from the group consisting of oxygenates, associated gas, unassociated gas, light gas from claim 10, and combinations thereof into a high shear device comprising at least one rotor and at least one stator; and
rotating the rotor to provide a tip speed of at least 22.9 m/s.

13. The method of claim 12 wherein the API gravity is increased by a factor of at least 1.5.

14. A system for producing value-added product from refinery-related gas, the system comprising:
at least one high shear device comprising at least one rotor and at least one complementarily-shaped stator, configured to produce a dispersion comprising bubbles of refinery-related gas in a liquid carrier;
apparatus for the production of a refinery-related gas comprising one or more of C1-C8 compounds; and
a pump configured for delivering a liquid stream comprising the liquid carrier to the high shear device.

15. The system of claim 14 further comprising a vessel coupled to said high shear device, said vessel configured for receiving the dispersion from said high shear device.

16. The system of claim 14 wherein the at least one rotor is rotatable at a tip speed of at least 22.9 m/s (4,500 ft/min), wherein the tip speed is defined as πDn, where D is the diameter of the rotor and n is the frequency of revolution.

17. The system of claim 14 wherein the at least one rotor is separated from the at least one stator by a shear gap in the range of from in the range of from about 0.02 mm to about 5 mm, wherein the shear gap is the minimum distance between the at least one rotor and the at least one stator.

18. The system of claim 14 wherein the at least one rotor is able to provide shear rate of at least 20,000 s⁻¹ during operation, wherein the shear rate is defined as the tip speed divided by the shear gap, and wherein the tip speed is defined as πDn, where D is the diameter of the rotor and n is the frequency of revolution.

19. The system of claim 14 comprising more than one high shear device.

20. The system of claim 14 wherein the high shear device comprises at least two generators, wherein each generator comprises a rotor and a complementarily-shaped stator.

21. The system of claim 14 wherein apparatus for the production of refinery-related gas comprises a cracker configured for breaking organic molecules into simpler molecules.

22. The system of claim 14 wherein the apparatus for the production of refinery-related gas comprises an oil refinery or some components thereof, a fossil fuel burning facility or some components thereof.

23. The system of claim 22 wherein the fossil fuel burning facility is a power plant or a power station.
